(19) United States
(12) Patent Application Publication
(10) Pub. No.: US 2017/0049554 A1
(43) Pub. Date: Feb. 23, 2017

(54) METHODS AND DEVICES FOR TREATING A HYPER-INFLATED LUNG

(71) Applicant: Soffio Medical, Inc., Boston, MA (US)

(72) Inventors: Jianmin LI, Lexington, MA (US); George BOURNE, Boston, MA (US); Mark GELFAND, New York, NY (US); Howard LEVIN, Teaneck, NJ (US); Michael BARENBOYM, Bedford, MA (US); Ary CHERNOMORSKY, Walnut Crieek, CA (US); Benjamin David BELL, Shrewsbury, MA (US); Gerhard Andrew FOELSCHE, Rehoboth, MA (US); Mark LEUNG, Duncan (CA)

(73) Assignee: Soffio Medical Inc., Boston, MA (US)

(21) Appl. No.: 15/307,727

(22) PCT Filed: Apr. 30, 2015
(86) PCT No.: PCT/US2015/028481

§ 371 Date: Oct. 28, 2016

Related U.S. Application Data
(60) Provisional application No. 61/986,270, filed on Apr. 30, 2014.
(60) Provisional application No. 62/088,881, filed on Dec. 8, 2014.
(60) Provisional application No. 62/108,040, filed on Jan. 26, 2015.

(51) Int. Cl. A61F 2/04; A61M 39/02

(52) U.S. Cl. CPC A61F 2/04; A61M 39/0247; A61F 2002/043; A61M 2039/0252

(57) ABSTRACT

A method and device to improve lung function in a patient having restricted ventilation. The device may include an implantable airway bypass device that relieves trapped air. The method may include a treatment procedure that minimizes irritation of tissue to control healing processes.

### RELATED APPLICATIONS

This application claims priority to U.S. provisional application Nos. 61/986,270 filed Apr. 30, 2014; 62/088,881 filed Dec. 8, 2014 and 62/108,040 filed Jan. 26, 2015, and the entirety of each of these applications is incorporated by reference.

### BACKGROUND

The present disclosure is directed generally to implantable medical devices to improve chest mechanics in diseased patients by partially bypassing natural airways. The methods and devices disclosed herein may be configured to create alternative expiratory passages for air trapped in the emphysematous lung by draining the lung parenchyma, thereby establishing communication between the alveoli and/or other spaces with trapped air and the external environment. Improvements over previous devices may include less invasive treatment, avoiding surgery and large area pleurodesis, minimizing disturbance and irritation of lung tissue to minimize inflammation or damage to untargeted areas of the lung and chest, improved control of healing processes, and establishing long-term patency of artificial air passages.

Disease of the lung such as Chronic Obstructive Pulmonary Disorder (COPD), emphysema, chronic bronchitis, and asthma may manifest with abnormally high resistance to airflow in an air pathway of the respiratory system. Homogeneous obstructive lung disease, also known as diffuse lung emphysema, is particularly difficult to treat and currently has few treatment options. Patients with pulmonary emphysema are unable to exhale appropriately, which leads to lung hyperinflation, which involves air trapping or excessive residual volume of air trapped in at least a portion of the lungs. The debilitating effects of the hyperinflation are extreme respiratory effort, the inability to conduct gas exchanges in satisfactory proportions, severe limitations of exercise ability, feelings of dyspnea and associated anxiety. Although optimal pharmacological and/or other medical therapies work well in the earlier stages of the disease, as it progresses, theses therapies become increasingly less effective. For these patients, the standard of care is surgical treatment involving lung volume reduction surgery, lung transplantation or both.

It has been observed in prior art and is generally accepted by clinicians that respiratory impairment in emphysema has an important ‘mechanical’ component. Destruction of pulmonary parenchyma causes compounding disadvantages of a decreased mass of functional lung tissue decreasing the amount of gas exchange, and a loss in elastic recoil and hence the inability to equally or substantially completely exhale the same amount of air that was inhaled on the previous breath. This leads to the typical hyperexpansion of the chest with a flattened diaphragm, widened intercostal spaces, and horizontal ribs, resulting in increased effort to breath and dyspnea. When the destruction and hyperexpansion occur in a nonuniform manner, the most diseased lung tissue can expand to crowd the relatively less diseased or even normal lung tissue further reducing lung function by preventing optimal ventilation of the less diseased or normal lung. Lung volume reduction surgery (LVRS) with surgical removal of the most affected lung regions conceptually would allow the relatively spared part of the remaining lung to function in mechanically improved conditions.

The majority of prior art in the mechanical approaches to emphysema addressed the opportunity presented by this non-uniform parenchymal destruction: removal of the parts of the lung most effected by the disease while letting the remaining lung to function normally, e.g., expand in a satisfactory manner, and improve the overall elastic recoil of the chest cavity. However, formerly attempted solutions have shown difficulties with long term device performance for example caused by tissue ingrowth, occlusion by naturally occurring secretions such as mucus or other secretions resulting from the heightened pro-inflammatory state in COPD, excessive bleeding, or rejection of an implant by the body.

### SUMMARY

Systems, methods and devices have been conceived and are disclosed herein for improving the mechanics of a diseased lung of a patient by implanting one or more natural airway bypass ventilation devices in a lung. For example, the patient may suffer from COPD, emphysema, chronic bronchitis, or asthma. A natural airway bypass device may comprise a pressure relief device connecting lung parenchyma distal to abnormally high resistance airways to the atmosphere.

Furthermore, devices and methods have been conceived and are disclosed herein for reducing residual volume and hyperinflation preventing the lung from hyper-inflating, and relieving symptoms of dyspnea and anxiety. The devices and methods may be configured to control healing processes at the implant site so that the device performance is maintained. The devices and methods may be configured to allow healing processes such as scarring and tissue growth to commence following implantation in a controlled manner that does not interfere with device performance over a long term.

A device for natural airway bypass of a diseased lung has been conceived and is disclosed herein that comprises, for example, a distal region and a proximal region, wherein the distal region comprises an air intake component and an expandable structure wherein the device is configured to hold the air intake component within a space in lung tissue created by an expandable structure, a conduit connecting the air intake component to the proximal region, and a strain relief member connecting the conduit to the air intake component, wherein a lumen in the conduit fluidly communicates between the air intake component and the proximal region.

A method and device for treating a hyper-inflated lung has been conceived and is disclosed there that comprises, for example, creating a space within the lung that is connected to a larger volume of the lung by collateral ventilation pathways, and providing an airway bypass pathway from the space to atmosphere. The airway bypass pathway may have a flow resistance that is low enough to allow air to flow. The method and device may be configured to minimize conditions for tissue regrowth. This may involve creating the space gradually, applying biologically active agents, or minimizing tissue irritation, inflammation, or friction between the device and lung parenchyma.

### BRIEF DESCRIPTION OF THE DRAWINGS

FIGS. 1 to 5 are schematic illustrations of chest anatomy showing a natural airway bypass device implanted in a lung.

FIG. 6 is a schematic illustration of a distal region of a natural airway bypass device implanted in a lung and partially deployed with a balloon catheter.

FIG. 7 is a schematic illustration of a distal region of a natural airway bypass device implanted in a lung and partially deployed with a pull wire.

FIG. 8 is a schematic illustration of a distal region of a natural airway bypass device partially expanded and compliant to varying lung tissues.

FIGS. 9 to 20 are schematic illustrations of a port device during implantation.

FIGS. 21 to 24 are schematic illustrations of an air collection device during implantation.

FIGS. 25 to 28 are schematic illustrations of a natural airway bypass system implanted in a chest wall and lung of a patient.

FIG. 29A is a schematic illustration of a natural airway bypass system having a membrane layer implanted in a chest wall and lung of a patient.

FIGS. 29B, 29C, and 29D are schematic illustrations of cross sections of a natural airway bypass system having a membrane layer.

FIG. 30 is a schematic illustration of a natural airway bypass system having a membrane layer.

FIGS. 31A and 31B are schematic illustrations of membrane layer orifices.

FIG. 32 is a schematic illustration of a natural airway bypass system having a membrane layer.

FIGS. 33 to 35B are schematic illustrations of a natural airway bypass system implanted in a chest wall and lung of a patient.

FIGS. 36A to 36D are schematic illustrations of a port device comprising a disc-shaped internal flange.

FIG. 37 is a schematic illustration of an internal flange having multiple petals.

FIG. 38A to 38D are schematic illustrations of an internal flange made from expanding foam.

FIGS. 39A to 39C are schematic illustrations of an internal flange comprising elastic cones.

FIGS. 40A to 40C are schematic illustrations of an internal flange comprising a spring mesh.

FIGS. 41A to 42D are schematic illustrations of an internal flange comprising multiple petals made from elastic wire loops and a flexible membrane.

### DETAILED DESCRIPTION

Systems, methods and devices are described herein for improving the mechanics of a diseased lung of a patient by implanting an airway bypass device that relieves pressure within the lung.

A method may comprise creating a small space in a lung that is fluidically connected to a larger volume of the lung through a natural phenomenon called collateral ventilation wherein air in the larger volume can flow to the created space. A natural airway bypass device may allow air to escape from the relatively small space created in the lung and thus relieve air trapped in the larger volume of lung that is connected to the space by collateral ventilation. Furthermore, devices and methods are disclosed for relieving hyperinflation of the lung having restricted air flow, for example due to COPD, emphysema or chronic bronchitis, and relieving symptoms of dyspnea and anxiety and improving quality of life.

The exact mechanism of collateral ventilation is also somewhat unclear and often debated. Candidate air pathways for collateral ventilation include the interalveolar pores of Kohn, the bronchioloalveolar communications of Lambert, and the interbronchiolar pathways of Martin.

COPD is characterized by slow or inefficient, sluggish flow of gas, e.g., air, exiting and emptying of alveoli. The air becomes trapped in the lung. The nature of air trapping is in a new breath being initiated before the exhalation of substantially all of the air inhaled on the previous breath is completed. An abnormally high amount of air is withheld in the lung, for example in the alveoli and alveoli ducts and bronchioles. These small air filled cavities are distal to areas of increased resistance to airflow that cause slow expiration. They are in fluid communication with each other, which enables the invention to empty the entire lobe or entire lung through one or few artificial channels.

A device and method of treatment have been conceived that allow auxiliary ventilation of a lung (e.g. enhanced, more complete or faster exhalation, pressure relief, reduction of residual volume) from small air-filled spaces where air is trapped. A device and method may allow air to pass from a first position within the lung to a second position. The first position may be an area of the lung that has higher pressure relative to atmosphere at the end of a natural expiration period of the breath. The second position may be to atmosphere, for example an exit port may be positioned on the surface or outside of a patient's body. Alternatively the second position may be within the natural airways of the patient's pulmonary system that has an air pathway to atmosphere that is less restricted, for example a location within the lung or bronchus.

Voids and low tissue density areas in the lung receive collateral ventilation and present a suitable target area for the implantation of the distal region of the device comprising an air intake component. However, a different area of the lung tissue interface may be better suited for sealing of the crossing of the pleura and the chest wall. Connecting tubing that is flexible and biocompatible may help placement of these components of the invention in the most suitable anatomic positions. The area best suited for the collection device implantation may be determined with the aid of high resolution CTA. Devices designed to determine collateral ventilation may help confirm that a particular area of a lung in a particular patient is a good candidate. The term ‘emphysema’ is generally used in a morphological sense, and therefore imaging modalities have an important role in diagnosing this disease. In particular, high resolution computed tomography (HRCT) is a reliable tool for demonstrating the pathology of emphysema, even in subtle changes within secondary pulmonary lobules. Among these morphologic changes to lung parenchyma, a particular pattern of destruction of the lung parenchyma, commonly referred to as pulmonary emphysema, is defined as “an abnormal permanent enlargement of the air space distal to the terminal bronchioles, accompanied by destruction of the alveolar walls, and without obvious fibrosis”. This pattern of destruction also creates an opportunity for therapy with the goal of reducing air trapping in the air spaces distal to airway obstruction or constriction. The opportunity lays in the dramatically increased natural collateral air flow and in the presence of low density, poorly vascularized areas of the lung where it may be feasible to create a space to collect air without a serious risk of bleeding or device failure due to closure by tissue ingrowth or scar tissue. This is expected since poor vascular blood supply results in less aggressive tissue growth in response to the initial injury.

The method and device may comprise an implantable device, or a partially implantable device, and be configured to minimize tissue regrowth that interferes with device performance, to minimize device rejection, to create a cavity in lung parenchyma, to transport fluid (e.g. air) from a position within the cavity to a second position of lower pressure (e.g. atmosphere).

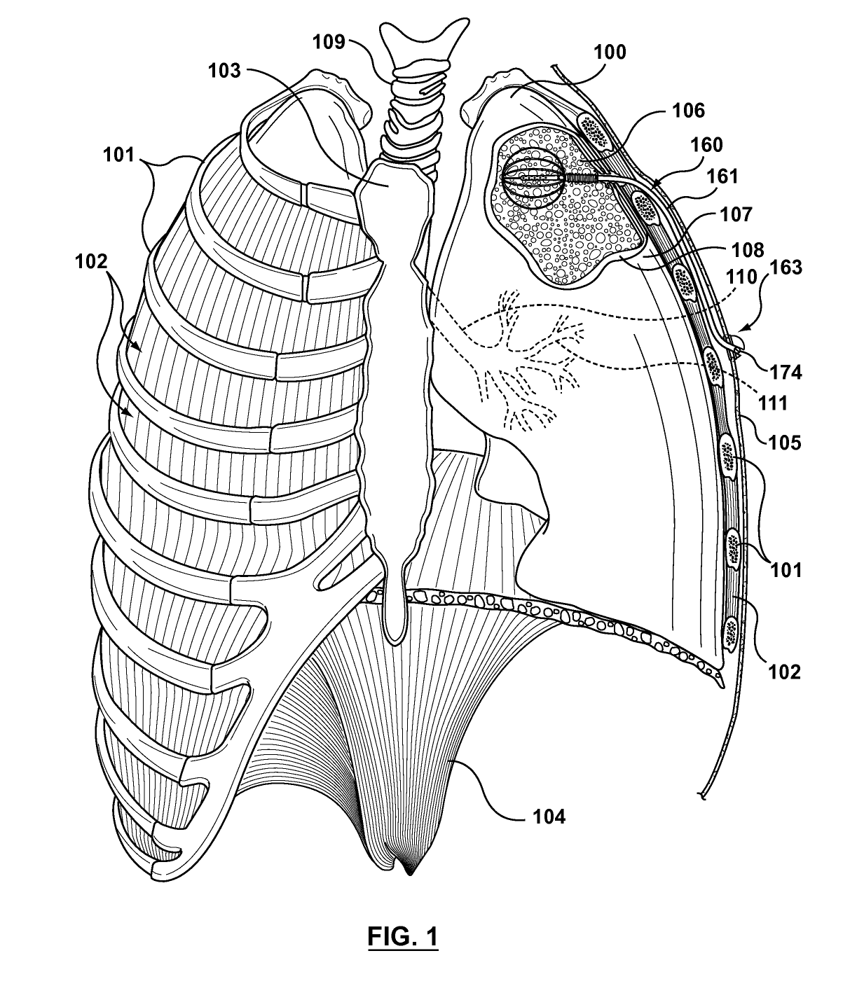

An embodiment of the present invention is shown in FIGS. 1 to 5. FIG. 1 is a schematic illustration of various layers of a patient's rib cage and thoracic cavity. Beneath the skin 105 is a rib cage formed by a vertebral column, ribs 101, and sternum 103. The rib cage surrounds a thoracic cavity, which contains structures of the respiratory system including a diaphragm 104, trachea 109, bronchi 110 and lungs 100. An inhalation is typically accomplished when the muscular diaphragm 104, at the floor of the thoracic cavity, contracts and flattens, while contraction of intercostal muscles 102 lift the rib cage up and out. These actions produce an increase in volume, and a resulting partial vacuum, or negative pressure, in the thoracic cavity, resulting in atmospheric pressure pushing air into the lungs, inflating them. In a healthy person, an exhalation results when the diaphragm 104 and intercostal muscles 102 relax, and elastic recoil of the rib cage and lungs 100 expels the air. In a patient having a disease such as COPD, emphysema, or chronic bronchitis a restriction in air pathways may make cause resistance to air flow and impede the ability of air to be expelled, in at least a portion of the lungs, upon muscle relaxation and elastic recoil of the rib cage. The inability to expel air from the restricted portion of the lung may result in a need for increased physical exertion to expel the air, increased residual volume, barrel chest syndrome, or feelings of dyspnea and anxiety. Lung parenchyma 106 is the tissue of the lung 100 involved in gas transfer from air to blood and includes alveoli, alveolar ducts and respiratory bronchioles.

In human anatomy, the pleural cavity is the potential space between the two pleurae of the lungs, namely the visceral and parietal pleurae. A pleura is a serous membrane which folds back onto itself to form a two-layered membrane structure. The thin space between the two pleural layers is known as the pleural cavity and normally contains a small amount of pleural fluid. The outer parietal pleura is attached to the chest wall. The inner visceral pleura covers the lungs and adjoining structures, via blood vessels, bronchi and nerves.

The pleural cavity, with its associated pleurae, aids optimal functioning of the lungs during breathing. The pleural cavity also contains pleural fluid, which allows the pleurae to slide effortlessly against each other during ventilation. Surface tension of the pleural fluid also leads to close apposition of the lung surfaces with the chest wall. This relationship allows for greater inflation of the alveoli during breathing. The pleural cavity transmits movements of the chest wall to the lungs, particularly during heavy breathing. This occurs because the closely apposed chest wall transmits pressures to the visceral pleural surface and hence to the lung.

A method of treatment has been conceived and is disclosed herein that, for example, connects the lung parenchyma to the atmosphere by passing through both layers of pleura. Sealing of the passage is required in order to prevent escape of the air into the pleural cavity and possible collapse of the lung. This seal is often referred to in this application as pleurodesis and shall be interpreted in the context of this disclosure. In common medical practice pleurodesis is described as a medical procedure in which the pleural space is artificially obliterated. It involves the adhesion of the two pleurae to each other over significant area of the lung often with the use of different agents that promote fibrosis, such as talc. As described in this disclosure it may refer to a creation of a seal, possibly with use of agents such as surgical glue, collagen, fibrin or alginate in order to prevent air escape around the puncture through the chest wall. Technically since two layers of pleura are fused in the process, it can be classified as pleurodesis with the limitation of it being local to the puncture site. The rest of the pleura, not adjacent to the puncture site, is desired to retain its normal qualities and function.

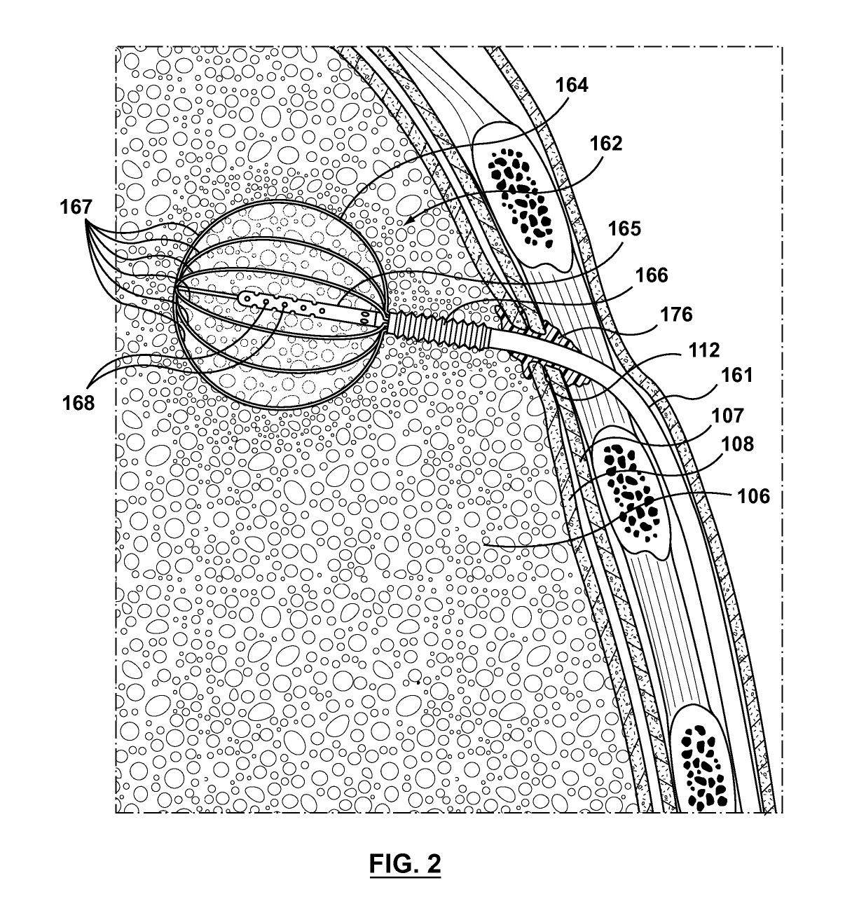

As shown in FIGS. 1 and 2 a distal portion of a natural airway bypass device 160 is implanted into the lung parenchyma 106. A proximal portion of the device 163 is positioned on the external surface of the patient's skin 105. The distal portion 162 is connected to the proximal portion 163 by a conduit 161 that passes out of the lung through a region of fusion 112 between the visceral pleura 108 and parietal pleura 107, passes beneath the skin 105 and exits the skin. Air or other fluids may pass from the distal region of the device 162 through the conduit and exit the proximal region of the device 163 external to the patient. A flow of air may be created by a pressure differential between a higher-pressure region in the lung to a lower-pressure at the proximal region of the device 163, which may be atmosphere. A pressure differential may be increased by further reducing pressure at the proximal region of the device, for example with a pump.

As shown in FIG. 2 a distal region 162 of a natural airway bypass device may comprise an expandable structure 164 surrounding an air intake component 165, which may be connected to conduit 161 via a flexible strain relief member 166. The expandable structure 164 creates an air collection space within lung parenchyma 106 and separates the lung parenchyma from the air intake component 165 and creates a surface area around the perimeter of the space that is substantially greater than the area of the ports in the air intake component 165 enhancing airflow from lung parenchyma to the space within the expandable structure 164 and through the air intake component 165. During the process of wound healing new tissue may proliferate into orifices and pores where it is in contact with an artificial material and especially where friction or other irritating forces are present between the tissue and such surfaces. By separating lung parenchyma from the air intake component the growth of new tissue into or over ports of the air intake component and occluding them may be reduced or avoided.

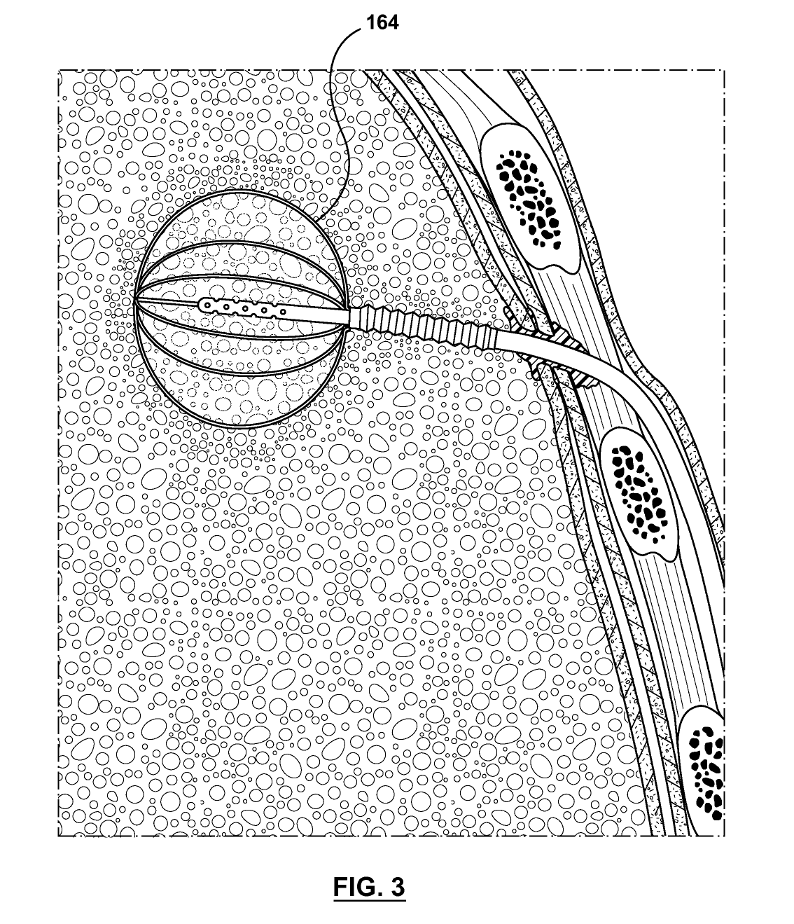

Furthermore, the expandable structure may be sufficiently compliant and flexible allowing it to substantially move, expand and contract along with lung parenchyma caused by breathing or coughing for example, thus minimizing friction, rubbing and potential tearing between lung parenchyma and the expanding structure, which may minimize or avoid irritation of the tissue decreasing a risk of excessive and prolonged, inflammation, scar formation or tissue regrowth. To illustrate this feature, for example, FIG. 2 may represent an exhaled state where FIG. 3 may represent an inhaled state in which the chest wall is expanded outward, the volume in the lung is expanded to pull air in and the expandable structure 164 expands in volume with the tissue surrounding it. Giant bullae in lungs are known to receive collateral ventilation from the rest of the lung (likely through alveolar ducts and alveoli that are embedded into their walls and were observed to remain open upon histological examination of an extracted lung) and tend to trap air and remain open and sufficiently void of fluids such as mucus. The expandable structure 164 may be used to create a similar effect of an air space connected to other portions of a lung through collateral ventilation.

The expandable structure 164 may comprise a scaffolding of struts that define the space within. The scaffolding may be for example a cage, a basket, a mesh, a weave, or a stent that can be delivered in a thin, undeployed state and expanded to a deployed state having an increased volume. As shown in FIG. 2, the expandable structure 164 comprises struts or fibers 167 deployed into a substantially spherical deployed state. The struts 167 may be made from a biocompatible, flexible material such as Nitinol, stainless steel, silicon, Pebax, PEEK, polypropylene, a composite of multiple materials or other biocompatible materials, such as biocompatible polymers. The expandable structure may be configured to have a sufficient quantity and sufficiently sized pores so that the fibers or struts stimulate fibrosis and tissue response and integrate into the tissue. For example, pore size may be between about 3 to 5 mm in diameter, or have a cross-sectional area between about 7 to 20 mm². Alternatively, a temporary structure may be used to support the air space and control a healing process and then the temporary structure may be removed or be biodegradable. Alternative embodiments comprise other deployed shapes such as funnel, torus, ovoid, cylindrical, or irregular shapes. The expandable structure may be compliant (e.g., applying very little to no pressure or force on the tissue) except when it is being deployed.

Alternatively a cavity can be created in the porous parenchyma of the lung by an expandable device such as a balloon or an injected bolus of biodegradable polymer. The expandable device can be then withdrawn and the support scaffolding deployed to control healing processes and prevent closure of the space.

The expandable structure (also referred to as expandable scaffold or cage) may be deployed gradually to control the healing process and minimize inflammation, bleeding and granulation. For example volume of the space created may be increased gradually over several hours, days or weeks by expanding in small increments until the fully deployed state is reached (e.g., 0.25 to 1.0 mL once every few days up to a fully deployed volume between about 3 to 20 mL (e.g., about 14 mL). The expandable structure may be deployed with a balloon (e.g., compliant balloon) inside the expandable structure.

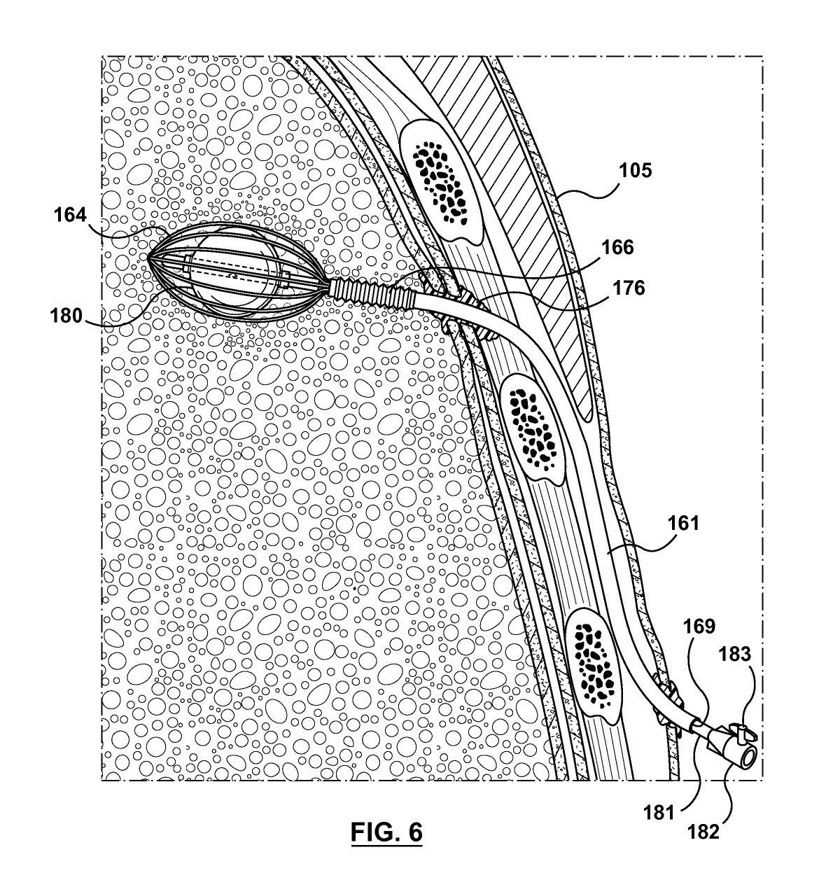

FIG. 6 shows the expandable structure 164 in a partially deployed state with a balloon 180 in the expandable structure. The balloon may be positioned on the end of a balloon catheter 181 that is inserted through a lumen 169 in the conduit 161. The proximal end of the balloon catheter may be configured to be inflated and comprise a valve. For example the balloon 180 may be inflated by injecting fluid (e.g., air, saline, x-ray contrast agent) into the balloon using a syringe attached to a fitting 182 on the proximal end of the balloon catheter. A valve 183 may be closed to hold the injectant in the balloon and maintain a state of deployment for a desired period of time. The state of deployment may be adjusted by injecting more injectant or retracting injectant from the balloon and the volume of the expandable structure, and thus space created in the lung parenchyma, may be increased in desired graduations. When the final deployed stated is reached the balloon may be left in place for a desired period of time to apply gentle pressure and maintain the volume while initial healing processes take place. After a desired period of time the balloon may be deflated and the balloon catheter 181 may be removed from the lumen 169 in the conduit 161. Optionally, a pressure sensor may be positioned in the balloon or balloon catheter to assess pressure asserted by the lung parenchyma on the expandable structure and balloon and as time passes after the final deployed volume is reached pressure may be decreased due to a healing process. A desired decrease in pressure may indicate a suitable stage for balloon deployment and removal. Optionally, a balloon for deploying the expandable structure or the expandable structure itself may be configured to deliver a biologically active compound. For example, a delivered compound may help to control a wound healing process while the balloon is gradually expanded. Examples of compounds that may help to control wound healing processes include anti-inflammatory drugs, which may reduce the secretion of cytokines, hyaluronic acid and other anti-adhesive agents, Streptokinase, which may reduce formation of fibrin, Tranilast, an anti-allergic agent used to prevent granulation tissue formation, collagenase or other protease for the enzymatic tissue disintegration. In one embodiment, a balloon may have micropores and the injectant used to deploy the balloon may contain a desired biologically active compound. Alternatively, a balloon or the expandable structure may be impregnated with a compound. Alternatively the expandable structure may have pores and a cavity containing a compound that is slowly released through the pores to the tissue.

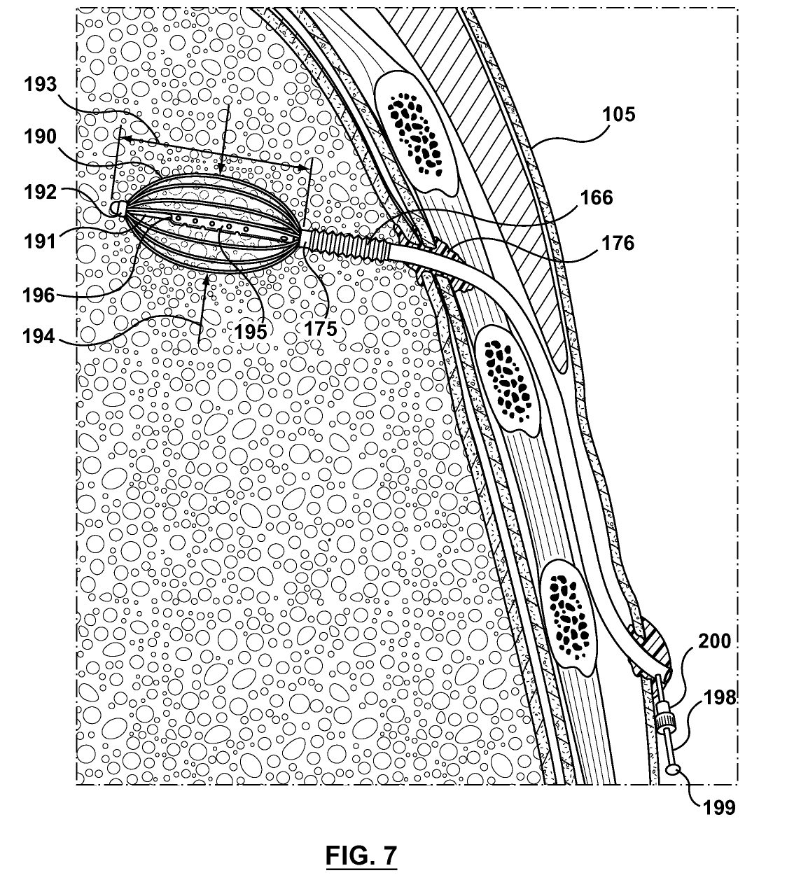

In an alternative embodiment, as shown in FIG. 7, an expandable structure 190 may be configured to deploy by applying tension to a pull wire 191 connected to a distal end of the expandable structure 192 to reduce the axial length 193 of the expandable structure 190. The expandable structure may be configured to respond to decreased axial length 193 by increasing in diameter 194. For example the struts 194 may be made from an elastic material such as Nitinol, or an expandable structure may be a wire weave or knitted or mesh structure or esophagus stent-like structure. In the embodiment shown in FIG. 7 the pull wire 191 passes through a pull wire hole 196 in an air intake component 195 and pass through a lumen in a conduit 197 to a proximal region of the device external to the patient where a proximal portion of the pull wire 198 is terminated in an end piece 199. A depth stopper 200 such as a collet may be used to adjust the tension on the pull wire 191 and thus the diameter 194 of the expandable structure 190 and the volume of the space created by the expandable structure. The position of the depth stopper 200 on the proximal portion of the pull wire 181 may indicate the degree of deployment of the expandable structure 190.

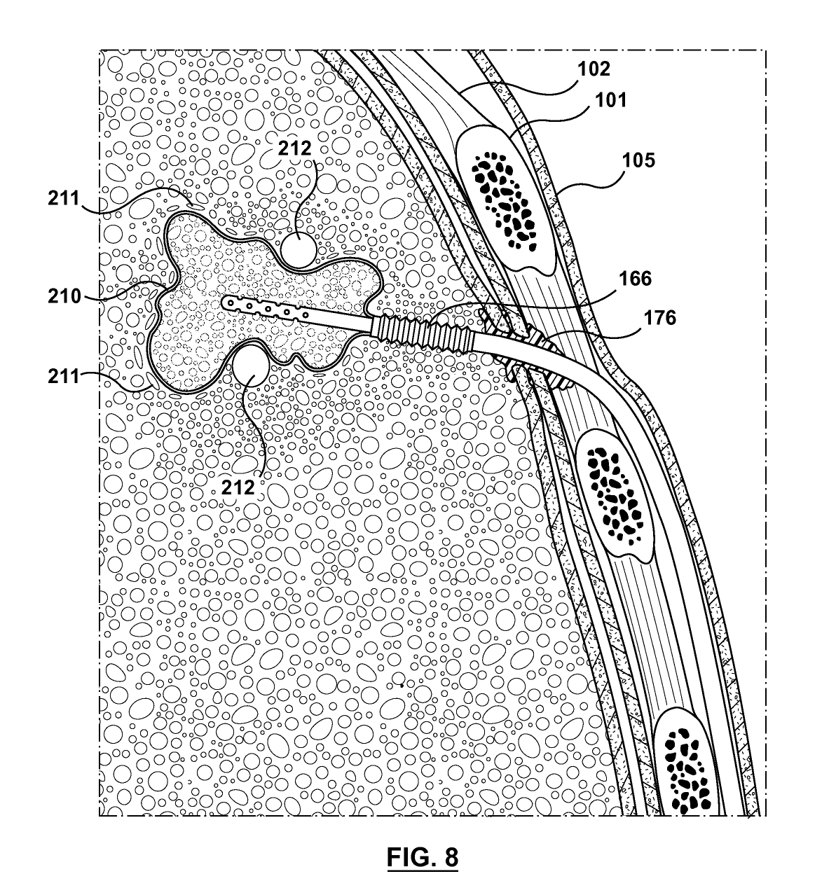

Alternatively, as shown in FIG. 8 an expandable structure 210 may create a space by expanding into a deployed shape that is dictated by physical features of the tissue surrounding the expandable structure. For example, a compliant expandable structure may expand by compressing tissue that is preferentially compressible or more flexible or softer 211 and deform around tissue that is less compressible or less flexible or harder 212. This characteristic may further improve the ability to create a space in lung tissue with minimal irritation and inflammation that could lead to tissue regrowth that could clog the air pathway through the natural airway bypass device. The proper pore size is selected to prevent or suppress tissue to close the bridge or close the pore opening. It is desired that the pores remain open after the tissue healing.

Other embodiments of devices or methods may be envisioned that create a space around an air intake component while creating an unfavorable environment in the space for tissue proliferation so air can pass unobstructed by tissue regrowth into the air intake component and out of the lung. Above examples gradually increase volume of a space created by inserting an expandable structure then altering its shape. Alternatively, volume may be gradually increased by gradually introducing a greater portion of a space creating device.

A distal region of a natural airway bypass device 160 may optionally comprise an energy delivery element to deliver energy such as electric, thermal, mechanical, or acoustic to facilitate control of healing processes or manipulate tissue characteristics such as its ability to recoil or expand and contract during breathing.

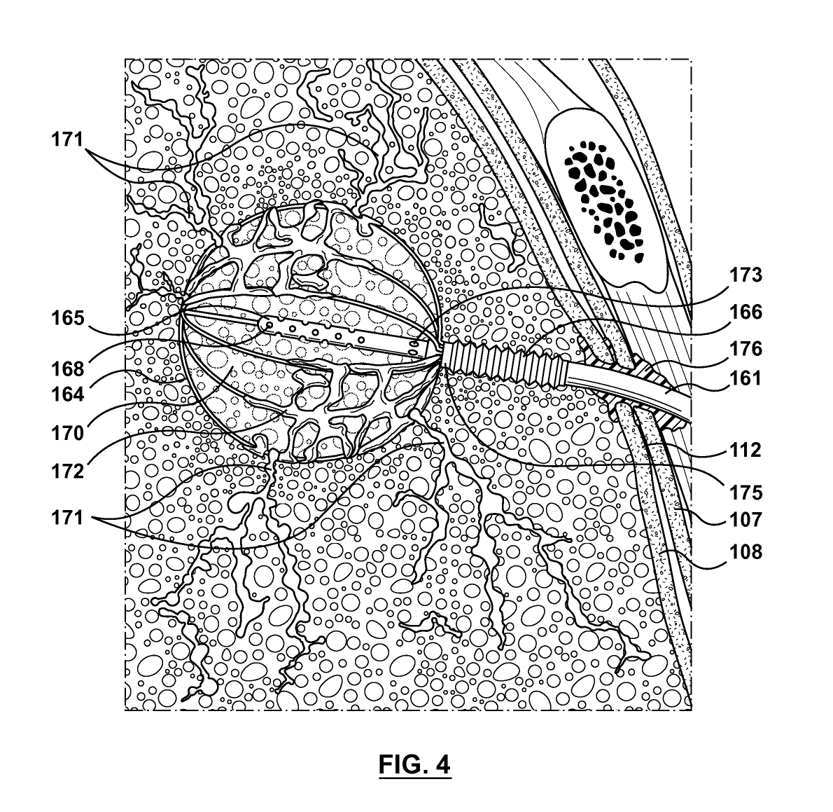

An expandable structure may help to maintain a space around the air intake component by reinforcing a perimeter of the space. Tissue surrounding the space may contain channels 171 or air pathways that connect the space 170 to parts of the lung containing trapped air as shown in FIG. 4. Healing processes may cause tissue to adhere or grow on to the expandable structure 164. However, a certain amount of tissue growth over the perimeter of the space 170 is permissible without impeding airflow in to the air intake component.

The air intake component 165 is a passageway for air to flow from the space 170 created in lung tissue by and expandable structure 164 to a lumen in the conduit 161. A natural airway bypass device may be configured to allow air to pass in one direction only. For example, a valve 174 positioned in the device such as at a proximal region 163 of lumen 169 or in an air intake component may allow air to flow out of the body only. Alternatively, a device may be configured to allow fluids (e.g. air, agents, saline) to pass from the distal region to the proximal region or from the proximal region to the distal region. For example, a device may be absent a valve or a valve may be removable or be bypassed or opened when desired. It may be desired to inject fluid from the proximal region of the device to the distal region of the device or space in the lung tissue. For example, it may be desired to inject a sterile fluid (e.g. air, mist, drug) to ensure tissue is not growing around air intake ports 168, to clean the space around the air intake component, or to deliver an agent to help maintain patency of the air passage ways, control healing processes, dilute air passageways, or treat infection or inflammation. Fluid injected to the distal region may pass through the air intake component 165 or alternatively through other ports 173.

The air intake component 165 (see FIG. 4) may comprise one or more ports 168 to at least one lumen in the conduit. For example, the combined area of the port(s) may be greater than the cross sectional area of the lumen in the conduit 161. The air intake port may be held in the space 170 created by the expandable structure 164 and away from the lung tissue surrounding the space to reduce the chance of tissue growing into the ports 168 or of the air intake component irritating the tissue. An air intake component may further comprise ports 173 connected to other lumens in the conduit 161 that may be used for example to deliver an agent to the space 170 for example to protect against infection or maintain patency of the space or airway connections to the lung. An air intake component may be made from a biocompatible polymer such as silicon, polyurethane or Pebax of soft durometer and may be an extruded tube. In the embodiment shown in FIG. 4, the air intake component 173 may be a Pebax tube with ports 168 machined or melted into the tube and edges may be rounded, for example during the forming process or after creation of the holes.

In the embodiment shown in FIG. 7 the air intake component 195 may comprise a pull wire hole 196 through which a pull wire may pass to control the expansion of expandable structure 190.

Alternatively, an air intake component may be on a distal portion of an elongate tube that is inserted into a lumen of a conduit. For example, the balloon catheter 181 shown in FIG. 6 may be used to gradually expand the space and then be removed from the conduit 161 and then an air intake component mounted on an elongate tube may be inserted through the lumen 169 of the conduit 161 and into the space of the expandable structure. The elongate tube may comprise a lumen for passage of air from the air intake component to the proximal region of the device. The elongate tube may also comprise a flexible, strain relief portion near the distal region of the tube that aligns with flexible strain relief member 166 of the conduit 161 so flexibility is maintained. The elongate tube containing the air intake component 165 may be inserted with a stylet to maintain rigidity until the air intake component is positioned in its desired location in the space and then the stylet may be removed. In this embodiment, the air intake component may be removed periodically and replaced with a clean one particularly to remove biofilm, mucus, granulation tissue or other matter deposited on the air intake component or in the lumen. The lumen 169 may also be used as an access port to assess the condition of the created space. For example, an endoscope may be inserted into the lumen to visually assess the space.

A natural airway bypass device as shown in FIGS. 1 to 4 and 6 to 8 may be configured for minimizing tissue regrowth that interferes with device performance and for minimizing device rejection. This may comprise minimizing friction between tissues in contact or in proximity with the device and the device itself. For example, the lung parenchyma is a soft tissue that undergoes motion with respect to the rib cage during breathing. Friction applied to tissue, for example tissue wounded by implanting a device, even in a carefully and gradually expanded implant as discussed herein, may cause inflammation or irritation that could lead to uncontrolled healing processes such as tissue regrowth that could interfere with device performance or instigate device rejection by the body. Minimizing friction around the device 160 where the conduit 161 passes through lung parenchyma to connect to the air intake component 165 and the expandable structure 164 may be accomplished with a flexible strain relief member 166. The strain relief member 166 may allow the distal region of the natural airway bypass device 162 to move with the lung tissue and apply very little force on the tissue. For example the strain relief member 166 may be a tube having a durometer that is of similar softness to the lung tissue. An embodiment of a strain relief member 166 as shown in FIG. 4 comprises a baffle made of soft material (e.g. silicone, Pebax, polyurethane) that can change in length or curvature when small forces are applied to the distal region by the lung tissue, allowing the distal region 162 to move freely with the lung tissue minimizing friction. Increased hardness of the strain relief 166 may be needed during implantation of the device 160. This may be accomplished by inserting a stylet of suitable hardness into a lumen of the conduit and the strain relief member and then removed after the device is implanted. Optionally, hardness of the strain relief member 166 may be gradually decreased after the device is implanted by replacing a hard stylet with stylets of incrementally softer durometer over time. This may improve control of healing processes. The strain relief member may comprise a gradual transition of durometer from the conduit 161 increasing in softness toward the distal end. For example, a transition of durometer may be accomplished by tapering wall thickness of a tube, thermal bonding multiple sections of material such as Pebax together that have varying durometer, multilayer co-extrusion of different materials and layers of various thicknesses or varying arrangement of baffles. The strain relief member 166 may be connected to the conduit 161 for example by thermal bonding or adhesive. The distal end of the strain relief member may be connected to the expandable structure 164 with a collar 175. In some embodiments such as shown in FIG. 7 an air intake component may be connected to the strain relief member with a collar 175 or by thermoforming or adhesive. The strain relief member may comprise a lumen in communication with a lumen in the conduit 161 for example for passage of air, fluid, catheters, replaceable sleeves, removable air intake catheters, or endoscopes.

In an alternative embodiment (not shown) a strain relief member may be configured to allow tissue to grow into its outer layer. For example, the outer layer may be made of a biocompatible mesh that cells can grow into. This may help to anchor the device, improve friction management and control healing processes.

A conduit 161 connects the distal region 162 to the proximal region 163 of the device. The conduit may pass directly out of the chest wall or as shown in FIG. 1 the conduit may pass beneath the skin a distance, which may reduce risk of infection in tissue around the distal region 162 of the device. The conduit 161 may be an elongate tube with at least one lumen in communication with the distal region 162 (e.g., via a lumen of a strain relief member) and the proximal region 163 and may be made of a biocompatible flexible material such as silicon, Pebax or other polymer. The lumen may be used for example for passage of air, fluid, catheters, replaceable sleeves, removable air intake catheters, or endoscopes. Multiple lumens may be present in the conduit. For example a second lumen may connect the distal region of the device 162 to the lymphatic system to drain collected fluid. A separate lumen may be used to deliver a drug from the proximal region 163 to a port 173 of the distal region as shown in FIG. 4. A replaceable inner sleeve may be inserted into the lumen of the conduit 161 to clean the passageway, for example to remove biofilm that may form within the sleeve over time. A replaceable inner sleeve may be replaced in a doctor's office as needed.

The conduit 161 may pass through a pleural obturator 176 as shown in FIG. 4. A region of fusion 112 may be made between the visceral pleura 108 and parietal pleura 107 using methods know in the art to avoid pneumothorax. A hole may be made in the fusion region 112 to gain access to the lung tissue. For example a hole may be cut with a scalpel to a desired size or a hole may be created by inserting a thin needle and gradually dilating the hole using a guidewire and dilators to minimize wounding of the tissue to control healing processes. A natural airway bypass device 160 may be inserted through the hole and a pleural obturator 176 or plug may be formed to seal the hole in the pleurodesis around the device. A pleural obturator 176 may be formed by injecting a sealant in to the space around the device in the area of the pleurodesis. The sealant may be injected as a fluid and may harden and adhere to the conduit 161 and tissue. Optionally, a pleural obturator may be formed prior to inserting a natural airway bypass device 160 which may help to form a pleurodesis. For example, a cannula may be inserted through the visceral pleura 108 and parietal pleura 107, a bioabsorbable anchor may be deployed through the cannula just inside the visceral pleura 108 and the cannula may be withdrawn slightly to pull the anchor against the visceral pleura. A collagen plug may be positioned on the outside of the parietal pleura 107 and a suture may pull the anchor and collagen plug toward one another to compress the two pleura.

Alternatively a pleural obturator may comprise a grommet-like device (e.g., made of silicon) that holds the pleurae together through which a natural airway bypass device may be inserted forming a tight seal between the obturator and device.

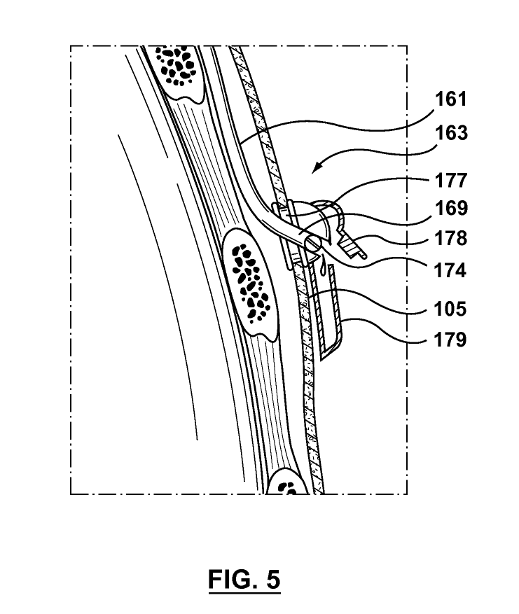

A plug 177 may also be positioned near the proximal region of the device 163 where the conduit 161 passes through the skin 105 as shown in FIG. 5. The plug may be a grommet that forms a seal around the conduit 161 and holds it within a hold in the skin 105. The plug may further comprise components on the exterior of the skin such as a cap 178 to cover the lumen 169, a fluid trap 179 to contain draining fluids and to facilitate cleaning, a drug delivery port (not shown) for example to connect to a syringe to deliver an agent into a drug delivery lumen and to the distal region 162 through drug a delivery port 173 (see FIG. 4), or flanges to adhere the plug to the skin of the patient.

In some embodiments a natural airway bypass device may be connected to a computerized controller that may be worn on the patient or may be a desktop controller that is periodically connected. A controller may apply energy to the device for example in the form of electrical energy, thermal energy, vibration, or acoustic energy to assist in control of healing processes. A controller may be used to control gradual expansion of an expandable structure, injection of biologically active substances, or device performance assessment.

A method of treatment may involve implanting a natural airway bypass device, such as the embodiment 160 shown in FIGS. 1 to 5, using techniques that will minimize or avoid tissue regrowth that interferes with device performance, minimize or avoid device rejection, and minimize or avoid irritation of tissue interacting with the device. The distal portion of the device comprising an air intake section may be placed in an upper (e.g. superior anatomical position) portion of a patient's lung, such as an upper lobe of a lung. Location may be chosen for placement of the distal portion of the device based on factors such as low tissue density, low blood flow, trapped air, presence of a bulla, or depth. The proximal portion of the device comprises an air escape section and may be placed exterior to the skin and may be inferior to the distal portion. Alternatively, the proximal portion of the device may be positioned within a patient's internal airway such as in a bronchus.

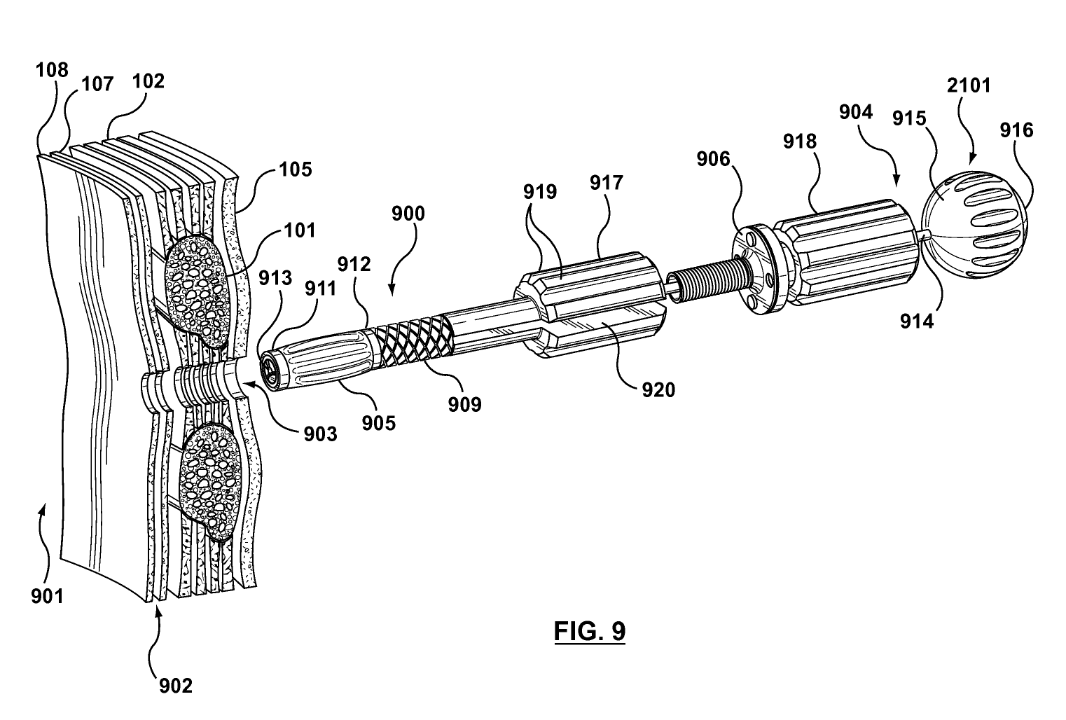

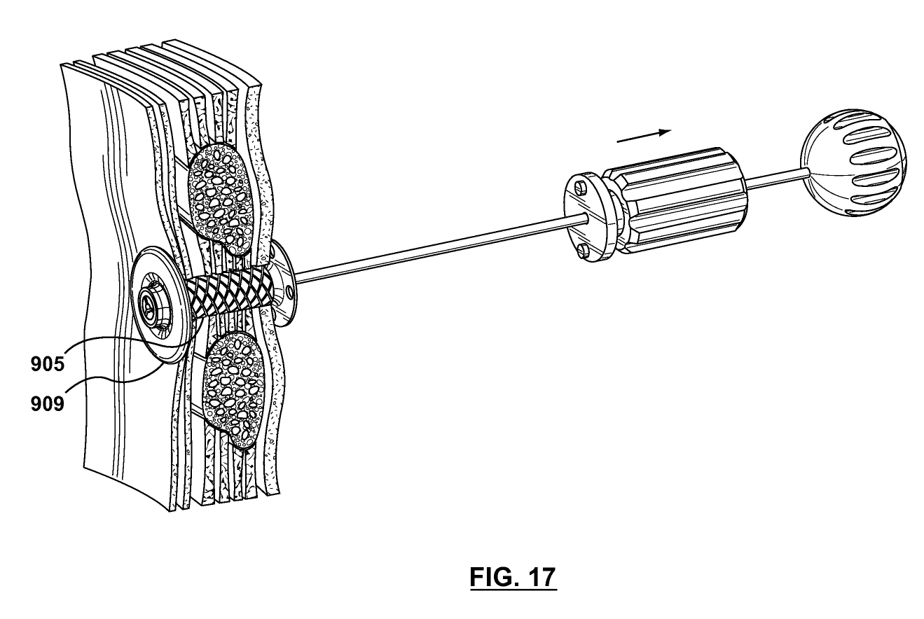

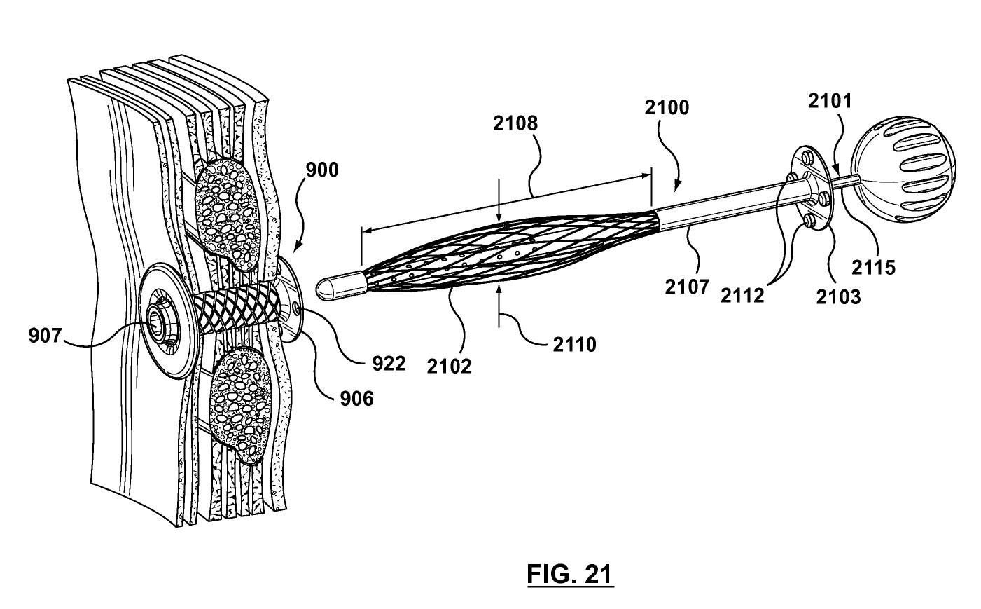

An alternative embodiment of a natural airway bypass system shown in FIGS. 9 to 25 comprises an implantable port 900 and an implantable air collection device 2100 (FIGS. 21 to 25). The port 900 provides the functions of creating an air seal between the pleural space 902, which is between the visceral pleura 108 and parietal pleura 107, and the passageway created through the chest wall to avoid pneumothorax (e.g. the seal may block fluid from passing from lung parenchyma or atmosphere to the pleural space 902); applying and maintaining pressure between the visceral 108 and parietal pleura 107 in an area around the port to form a pleurodesis; promoting fibrosis at an interface between the port tube 909 and adjacent tissue; forming a seal between the port 900 and skin 105; providing a channel configured to accommodate delivery of instruments or devices and a barrier between the channel and the tissues of the chest wall including visceral and parietal pleura to eliminate frictional or rubbing forces of the instruments or devices on the tissues of the chest wall thus minimizing undesired irritation, inflammation, or tissue growth; preventing closure of a passageway created through the chest wall that otherwise may be caused by tissue regrowth; or providing a connection to the air collection device 2100 to maintain its general position in lung parenchyma with respect to the port. The air collection device 2100 provides the functions of creating a space within lung parenchyma with a deployable scaffold 2102 (e.g., cage) wherein components of the deployable scaffold may be at least partially encapsulated by tissue while maintaining open pores sufficient to allow pressurized air to permeate from the lung into the space created by the scaffold; positioning an air collection tube 2104 having at least one opening within the space in the scaffold, wherein the surface area of the space created in lung parenchyma may be greater than the surface area of the opening(s) 2105 of the air collection tube (e.g. the surface area of the space created in lung parenchyma may be more than about 1.5, 2, 4, 10, 20, 30, 40, 50, 60, 70, 80, 90, 100 times the surface area of the opening(s)); maintaining distance between the at least one opening 2105 of the air collection tube 2104 and lung parenchyma sufficient to avoid occlusion of the at least one opening caused by tissue growth; or connecting the air collection device 2100 to the port 900.

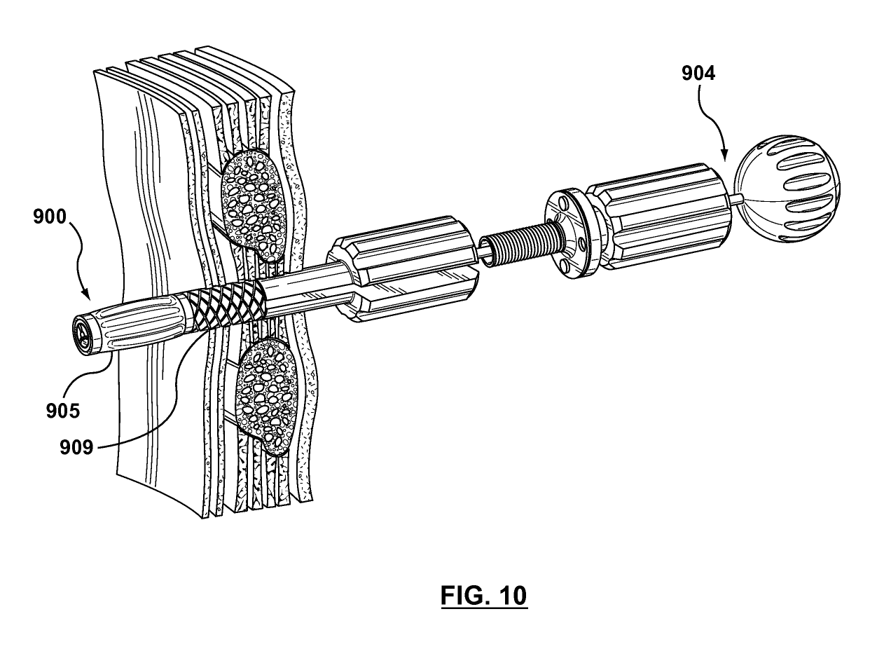

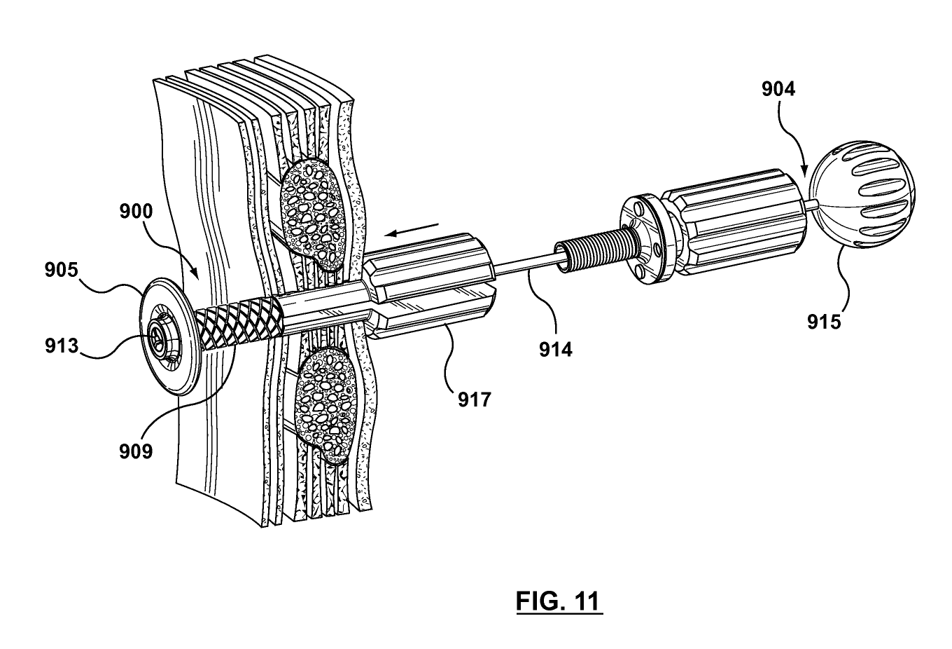

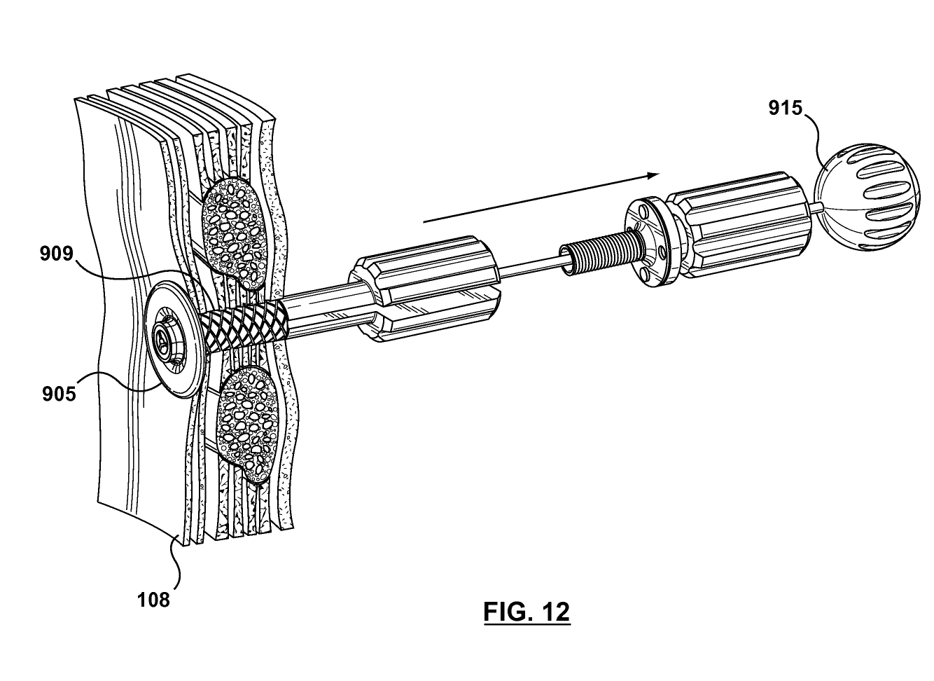

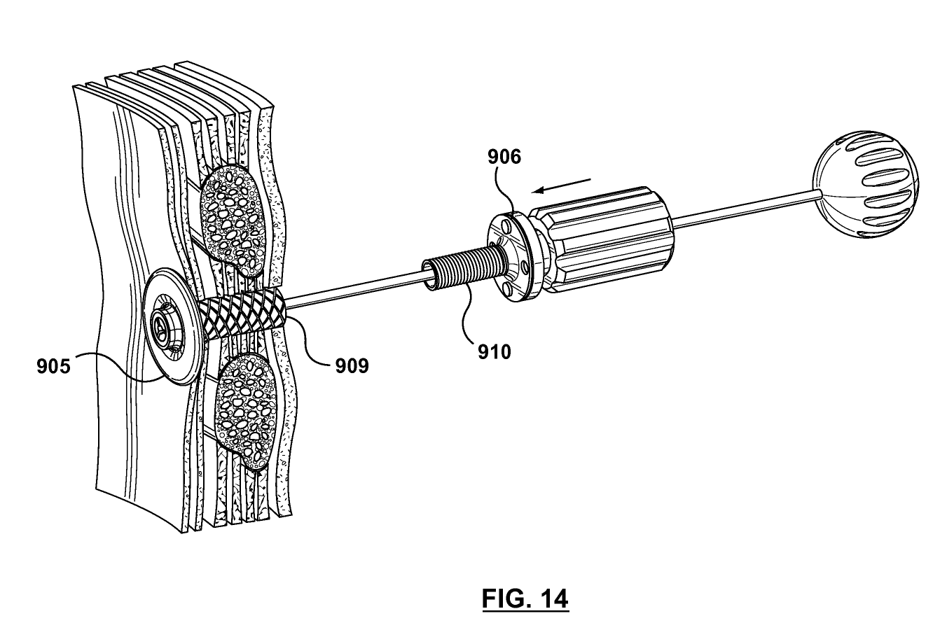

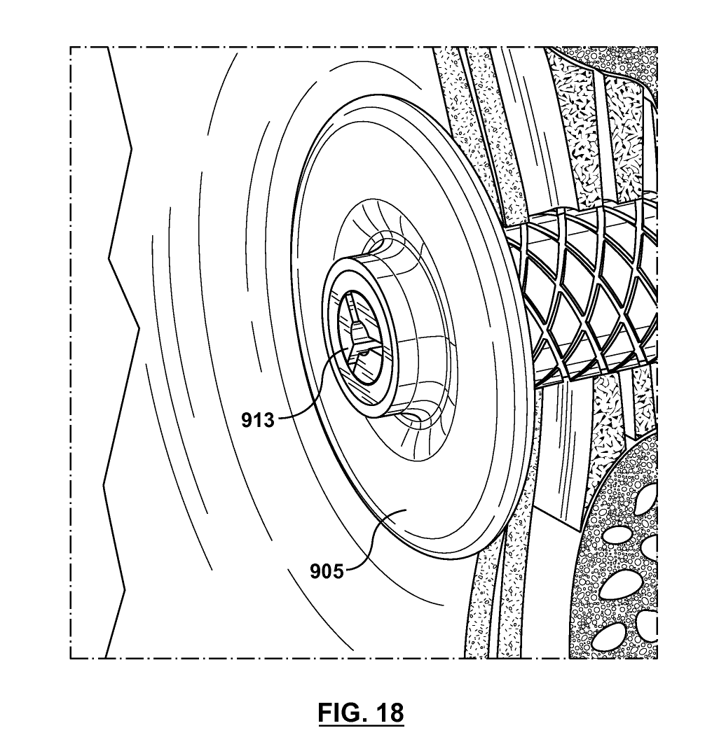

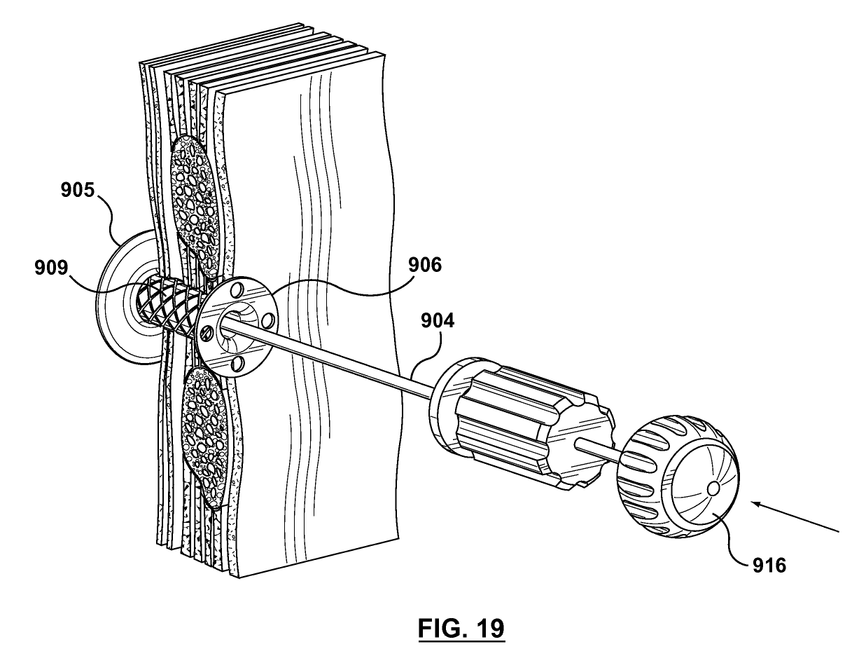

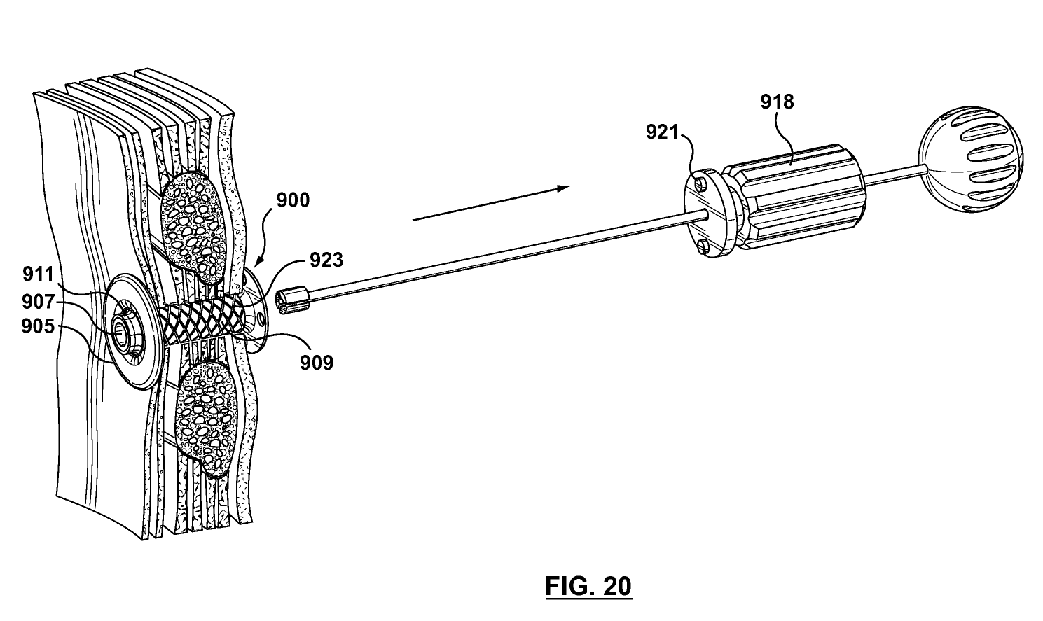

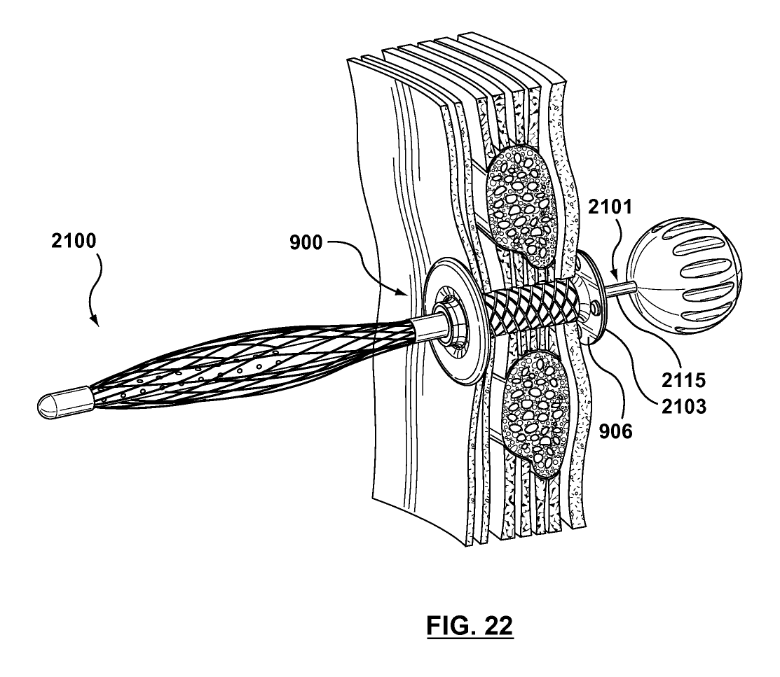

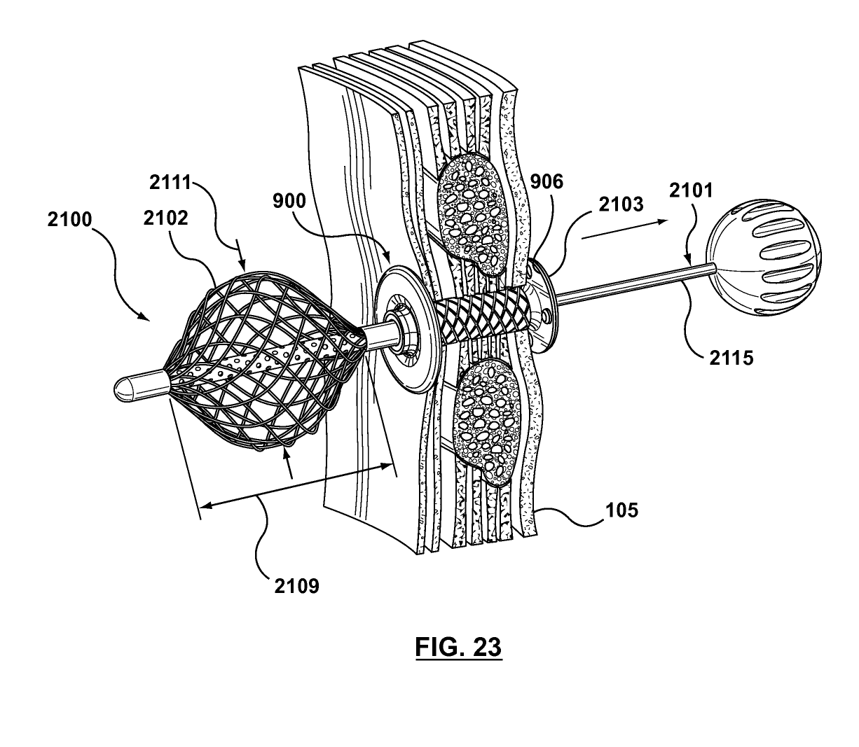

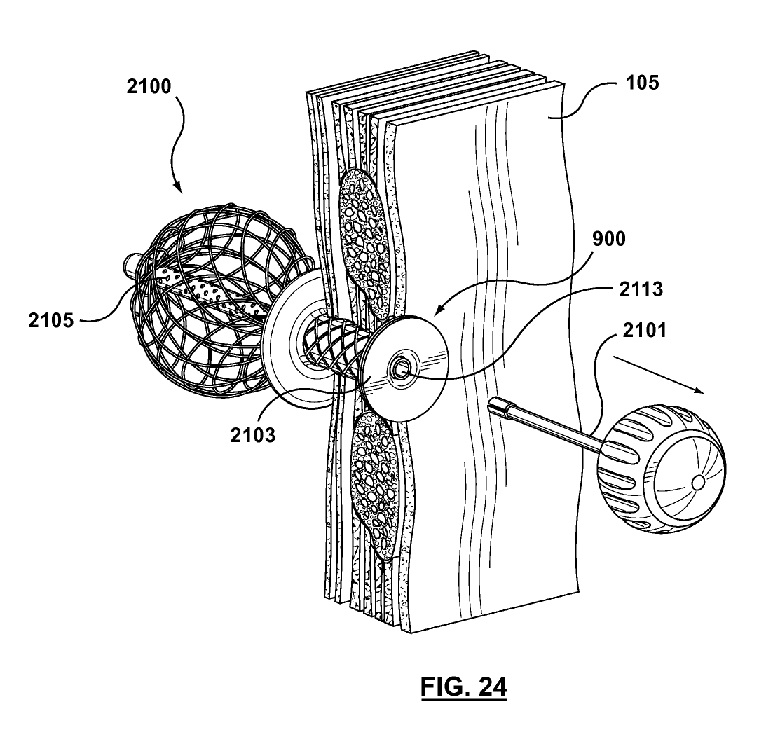

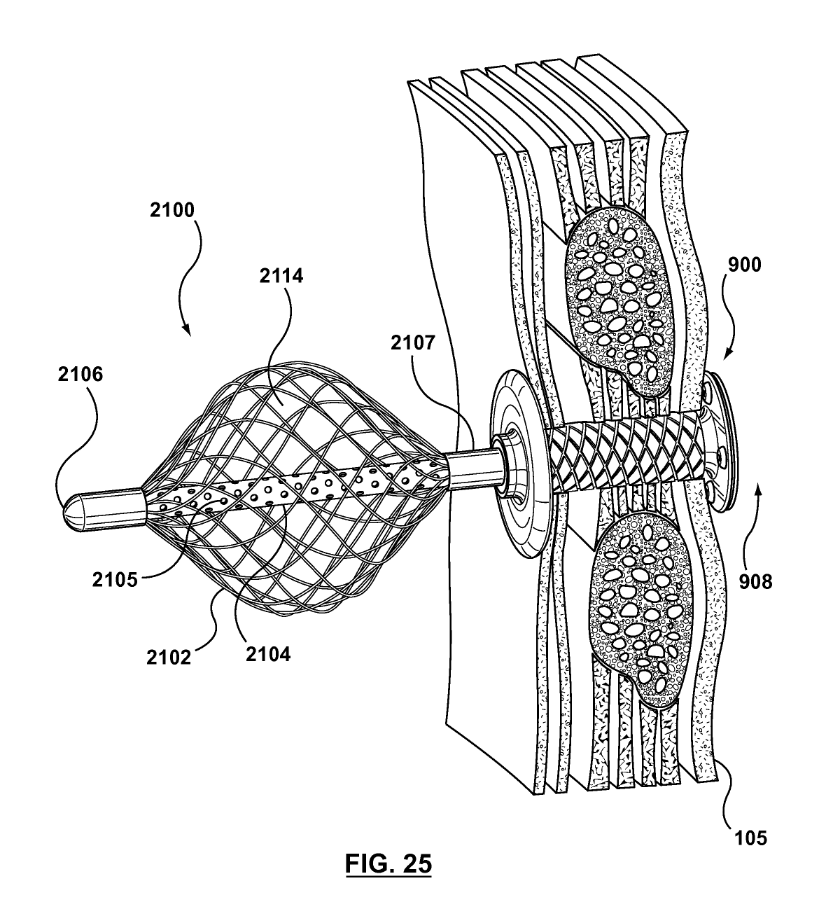

A method of use may comprise creating a passageway 903 through the chest wall consisting of skin 105, intercostal muscles 102 between ribs 101, parietal pleura 107, and visceral pleura 108 to a space within lung parenchyma on the internal side 901 of the visceral pleura. The position of the space within the lung parenchyma may be identified, for example using medical imaging technology such as CT scan, to comprise at least one of the following characteristics: low density lung tissue, upper (e.g., cranial) portion of a lung, comprising a bulla, collateral ventilation channels, or not containing major blood vessels that can may increase a risk of iatrogenic injury such as bleeding. The passageway 903 may be created using surgical techniques such as inserting a needle, inserting a guidewire through the needle, removing the needle, and inserting a coaxial dilator or set of dilators to open the passageway. A dilator set may comprise a tear away sheath. Alternatively, a similar device may be configured for creating a passageway. For example, an insertion tool may have a sharp tip (not shown) that may create a passageway while a port is being inserted. The method of use may further comprise inserting the port 900 in to the passageway 903, or an optional tear away dilator sheath (not shown) using and insertion tool 904 (FIG. 10); deploying an internal flange 905 of the port 900 (FIG. 11); applying pressure between the internal flange 905 and internal side of the visceral pleura 108 to press the visceral pleura 108 and parietal pleura 107 firmly together (FIG. 12) optionally removing the dilator tear away sheath (not shown); connecting an external flange 906 to the internal flange 905 (FIGS. 14 to 16); removing the insertion tool 904 (FIGS. 19 to 20); inserting an air collection device 2100 (also referred to as an air ventilation catheter or tube) in an undeployed state through a lumen 907 in the port 900 using an insertion tool 2101 and connecting an external flange 2103 of the air collection device 2100 to the external flange 906 of the port 900 (FIGS. 21 to 22); deploying a scaffold 2102 of the air collection device 2100 using the insertion tool 2101 (FIG. 23); and removing the insertion tool 2101 (FIG. 24) from the air collection device 2100. Remaining implanted in a patient's lung is a deployed scaffold 2102 that creates a cavity in lung parenchyma, a air collection tube 2104 held within the space created by the deployed scaffold 2102 and having at least one opening 2105 in fluid communication with a lumen in the air collection tube 2104 that is in fluid communication with external atmosphere 908 (FIG. 25). Trapped air in a patient's emphysematous lung at a pressure that is higher than the surrounding atmosphere 908 may pass from the lung parenchyma through the scaffold 2102 in to the space within the scaffold, through the opening(s) 2105, through the lumen of the air collection tube 2104 and be released to the atmosphere 908. Biomaterials such as fibrin may be injected into pleural space around the passageway to glue pleurae together to improve the seal. An endoscope may be delivered through the channel of the port device prior to delivering the air collection device to assess the function and placement of the port device or assess lung tissue.

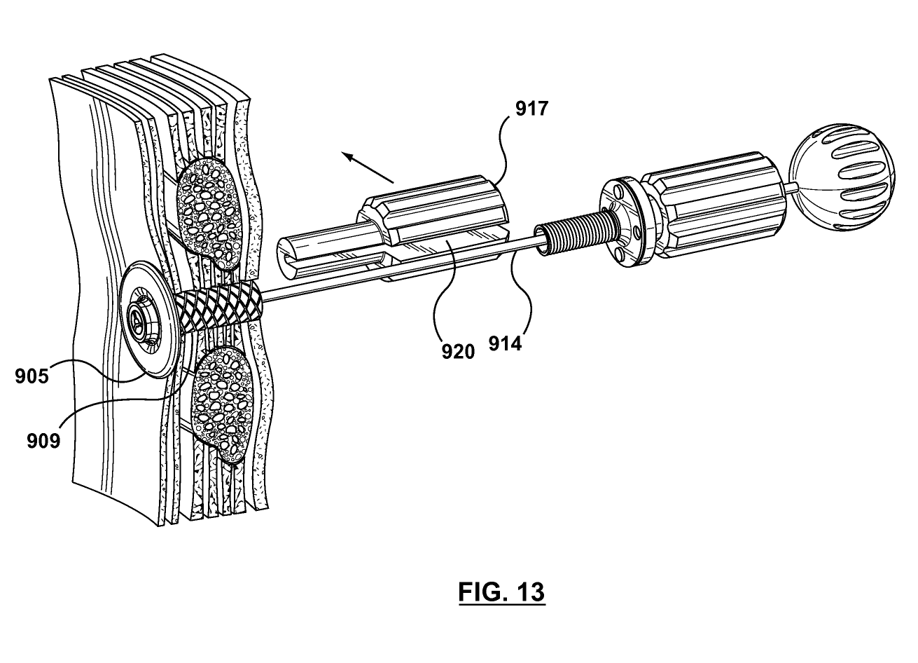

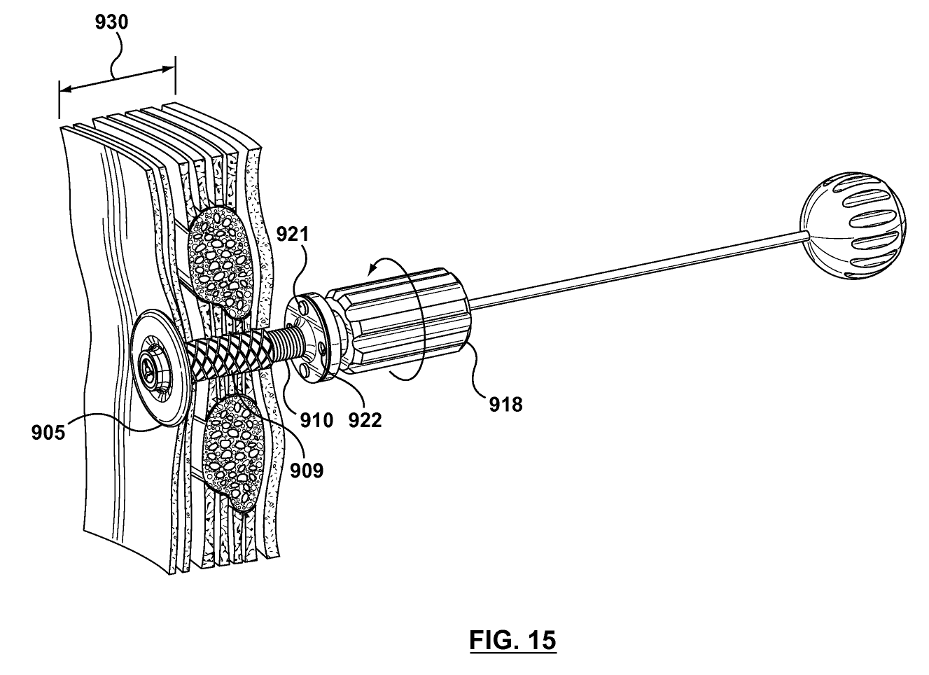

An insertion tool 2101 may be used to implant the port 900 and air collection device 1200. An insertion tool may comprise one single tool or a set of separate tools (e.g., a tool for implanting a port and a separate tool for implanting an air collection device). An insertion tool as shown being used in FIGS. 9 to 24 may comprise a lock mechanism 913 connected to a shaft 914, which is connected to a handle 915. The handle may comprise an actuator (e.g., a button, lever, knob, pull wire) 916 (FIG. 19) that disengages the lock mechanism 913. For example the actuator 916 may be connected to the lock mechanism 913 via a rod positioned in a lumen of the shaft 914. An insertion tool may also comprise a first slidable collar 917 used to deploy an internal flange 905 of the port 900 and a second slidable collar 918 used to connect an external flange 906 of the port 900 (see FIG. 9). Alternatively, an external flange may be configured with a surface (not shown) suitable for inserting by hand wherein a second slidable collar 918 may not be needed. The slidable collars 917 and 918 may have a lumen that is slightly larger than the shaft to allow the collars to slide over the shaft 914 and may be configured to be held and advanced by a user particularly wearing surgical gloves. For example, the slidable collars may comprise a gripable surface having ridges 919. For example, the ridges may be parallel to the axis of the shaft 914 as shown in FIG. 9 or perpendicular to the shaft (not shown). The first slidable collar 917 may further be configured to be removed from the insertion tool shaft 914, for example, a radial gap 920 as shown in FIG. 13. A second slidable collar 918 may be configured to connect the external flange 906 to the port tube 909. For example, as shown in FIG. 15 the second slidable collar 918 comprises tabs 921 that mate with holes 922 in the external flange allowing the second slidable collar 918 to transmit rotational motion to the external flange. Alternative configurations for mating a slidable collar 918 to an external flange and allowing transition of rotational motion may be envisioned.

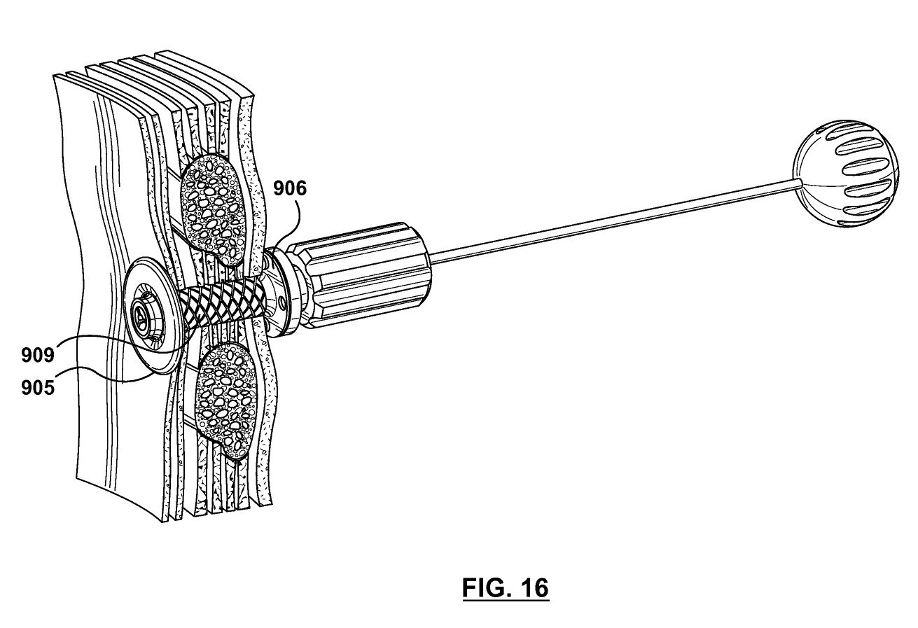

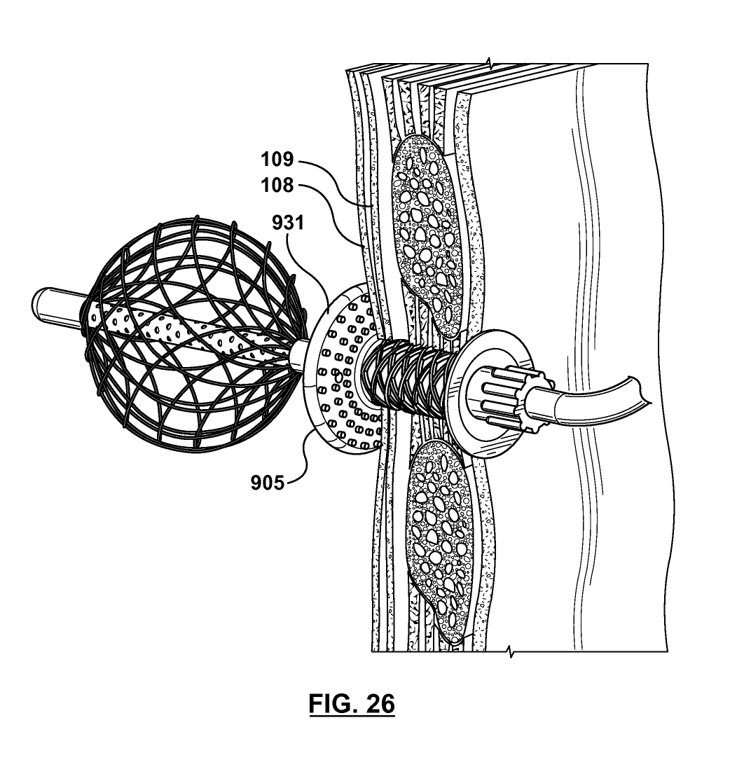

The port, as shown in an implanted configuration in FIG. 20 and during steps of implantation in FIGS. 9 to 19, may comprise a deployable internal flange 905 connected to a port tube 909. Other structures (e.g., balloon, linkages, that create a surface area in a deployed state that will not pass through a passageway 903 may be alternatives to an internal flange 905. The internal flange 905 may be configured to be deformed from an undeployed state (FIGS. 9 and 10), which may have a profile allowing it to pass though the passageway 903 or through a lumen of a dilator or sheath (e.g. having a diameter of about 2 to 3 mm, between about 1 and 15 mm, between about 2 to 5 mm) to a deployed state (FIG. 12) having a diameter of about 5 to 20 mm greater than the undeployed state (e.g., between about 5 to 50 mm, between about 10 to 20 mm, larger than an intercostal space through which the port is delivered). When the internal flange 905 is placed in contact with visceral pleura 108 and a pressure is applied (FIG. 12) an air tight seal is made between the internal flange 905 and the visceral pleura 108 and contact pressure is created between the visceral pleura and parietal pleura, which may develop in to a pleurodesis or which may control or avoid creation of a peurmothorax. The internal flange 905 may be made from an elastomer having a deployed shape such as a disk as shown in FIG. 12. A distal end of the elastomer material may be connected to distal collar 911 and a proximal end 912 of the elastomer material may be connected to a port tube 909 (FIG. 9). The distal collar 911 and port tube 909 may be made from a biocompatible material such as a polymer such as polyurethane or polypropylene. The distal collar 911 may engage with a lock mechanism 913 of an insertion tool 904 (FIG. 18). The distal collar 911 and the port tube 909 may be pushed toward one another thus decreasing axial length of the elastomer and increasing its diameter to transition from an undeployed state to a deployed state, for example by moving the lock mechanism 913 that is engaged with the distal collar 911 toward the port tube 909 by distally advancing the first sliding collar 917 with respect to the handle 916. The distal collar 911 may engage with the port tube 909, for example with a snap fit having audible or tactile confirmation, to maintain the internal flange 905 in a deployed state when the first slidable collar 917 is released. In alternative embodiments an internal flange may be configured to increase stress concentration, or concentrate pressure, applied to the visceral pleura to improve an air seal, improve grip or traction, or improve a seal between the visceral and parietal pleura. For example, as shown in FIG. 26, an internal flange 905 may comprise small protrusions or textured surface 931 on the surface meant to apply pressure to the visceral pleura 108. Alternatively, a configuration to concentrate pressure may comprise a protruding ring or concentric rings. Once deployed, a user may gently pull the insertion tool (e.g., by the handle 915) away from the chest wall to apply pressure between the internal flange 905 and the visceral pleura 108 (FIG. 12). The first slidable collar 917 may be removed from the insertion tool shaft (FIG. 13) and the external flange may be advanced to mate with the port tube 909 (FIG. 14). The external flange may be made from a biocompatible material for example molded from a polymer such as polyester or polypropylene and may comprise a tube 910 configured to securely mate with port tube 909 for example an external surface of the tube 910 may be treaded to screw into a threaded lumen of tube 909 (FIG. 15). Alternative mating mechanisms between tube 910 and tube 909 may be envisioned. Optionally, a mating mechanism may allow a length of the port 900 between internal flange 905 and external flange 906 to be adjustable or customizable to fit varying chest wall thicknesses. The external flange 906 may be advanced into the port tube 909 (e.g., screwed in to the tube 909) until a desired pressure is applied between the flanges 905 and 906 and structures of the chest wall to maintain pressure between the pleurae and maintain an airtight seal (FIG. 16). For example, the length of the port 900 between the internal 905 and external 906 flanges may be adjustable between about 2 to 8 cm. Alternatively, the length may be non-adjustable but chosen to be suitable for a patient. When the insertion tool is removed by disengaging the locking mechanism 913 (FIGS. 19 to 20) a lumen 907 through the port 900 provides access to the patient's lung parenchyma. The lumen 907 may be formed at least in part by a hole in the external flange that communicates with a lumen in the external flange tube 910 that communicates with a lumen through distal collar 911. The lumen 907 may also be formed at least in part by a lumen in port tube 909. The external surface of the port tube 909 that interfaces with tissues of the chest wall (e.g. a tissue interface surface) may be configured to allow tissue ingrowth. For example, a tissue ingrowth sheath 923 made from a synthetic mesh such as Polyethylene terephthalate (e.g., PET, Dacron®, Terylene®) may be affixed to the port tube 909. A tissue ingrowth sheath 923 may be cut to a desired length to accommodate port device having an adjustable length. Alternatively, the surface may have a porous or mesh-like texture incorporated in to the mold of the port tube 900. Controlled tissue ingrowth in to external surface of the port 900 may further secure the port in the chest wall, reduce irritation or reduce a prolonged healing process (e.g., production of granulation tissue, scabbing, inflammation), reduce uncontrolled tissue regrowth that may impede function of the device, reduce risk of infection, or improve the seal between tissue and the device.

An alternative embodiment of a port device (not shown) is configured to allow expansion and contraction of the length of the port device in response to motion of the chest wall while maintaining a pleural seal and minimizing irritation of tissues of the chest wall. For example, an internal flange may be resiliently flexible and be designed to apply sufficient sealing pressure to the visceral pleura over a range of chest wall motion. An internal flange may have a funnel or suction cup shape wherein the outer region of the flange applies pressure to the visceral pleura, the inner region connects to a channel through the port device, and the material between the outer region and inner region is elastically resilient to allow motion while applying pressure. An internal flange may be a sponge-like material. Port device may comprise an elastically resilient member holding an internal flange to an external flange that allows the distance between the flanges to expand or contract with movement of the chest wall while maintaining pressure on the pleurae.

An embodiment of an air collection device 2100, as shown in FIGS. 21 to 25, is configured to be inserted in an undeployed state through lumen 907 of the implanted port 900, connected to the port 900, and deployed in lung parenchyma. The air collection device 2100 may comprise a deployable scaffold 2102 that may be connected to a tube 2104 at its distal end, for example it may be held to the shaft with a distal end piece 2106 that has a rounded tip to reduce injury to the lung parenchyma as it is inserted. The scaffold 2102 may be connected to a sheath 2107 at its proximal end. The tube 2104 may slide telescopically within a lumen of the sheath 2107. In an undeployed state, the tube 2104 is fully extended giving the scaffold structure 2102 a first length 2108 and an undeployed diameter 2110 configured to pass through lumen 907 (FIG. 21). In a deployed state, the tube 2104 may be retracted into the sheath 2107 reducing the length of the scaffold 2102 to second length 2109 and increasing the diameter to a second diameter 2111 (FIG. 23). During implantation an insertion tool 2101, which may be the same or separate device as the insertion tool 904 used to implant the port 900, may have an actuatable lock mechanism at a distal end of its shaft 2115 that engages with the tube 2104 (e.g., via the distal end piece). Once the air collection device 2100 is delivered into the lung parenchyma and connected to the port 900, a user may hold the flange 2103 against the port's external flange 906 or chest wall while pulling the handle 2116, which transmits the pulling force down the shaft 2115 to the lock mechanism to the distal end piece 2106 causing the tube 2104 to slide in a lumen of sheath 2107 to deploy the scaffold 2102. When fully deployed the tube 2104 may be locked in position within the sheath 2107, for example with a snap fit, to hold the scaffold 2102 in a deployed state when force is released. The sheath 2107 may be connected to a flange 2103 that is configured to connect to external flange 906 of the port 900. For example, as shown the flange 2103 may have tabs 2112 that mate with holes 922 in the external flange 906 of the port 900 with a snap fit and audible click. Other configurations for connecting the sheath 2107 to the port 900 may be envisioned. Optionally, the air collection device 2100 may be configured to temporarily be connected to the port 900 so it may be removed or replaced. The tube 2104, also referred to as a venting catheter, may have at least one opening 2105 in fluid communication with a lumen in the tube 2104, which is in fluid communication with a lumen 2113 in sheath 2107 that may vent air to the atmosphere external to the skin 105. The sheath 2107 and tube 2104 within the sheath may be configured (e.g. with sufficient flexibility and length) to allow the expandable structure 2102 to move with lung tissue with respect to the port device 900 in the chest wall. For example the section of the sheath and tube between the internal flange and the scaffold structure may be in a range of about 5 mm to 15 mm and the material may be a soft durometer to allow bending which allows the expanded scaffold structure to move for example when a patient breaths, coughs, or sneezes to reduce risk of tissue trauma that might be greater with a rigid device. The tube 2104 may be made from a polymer such as Pebax and may have a hydrophobic coating such as a Teflon copolymer on its inner and outer surface that may reduce or prevent fluids from sticking or tissue from adhering to the tube to improve airflow through the tube. Alternatively, at least one component such as the tube 2104 may be made of a polymer having an additive that gives it a non-stick surface. For example, a polymer compound such as Endexo® may be used to make any of the components of the device to reduce or eliminate the ability of tissue to stick to the device. Alternatively, materials used to make the device may be heparin coated to reduce the chance of blood clotting on the surface. The deployable scaffold 2100, also referred to as a cage, may have a deployed shape such as a spheroid, bud shape, cone, cylinder, basket or other shapes that create a cavity in lung parenchyma that helps to maintain patency of opening(s) 2105. The scaffold may expand to a shape dependent on tissue density or varying resistive pressure applied by the tissue on the expanding scaffold. The cavity created by the scaffold 2100 may have a volume between about 4 cm³ and about 1500 cm³ (e.g., between about 4 and 180 cm³, 4 and 100 cm³, between about 10 and 20 cm³, between about 20 and 50 cm³, between about 50 and 100 cm³, between about 100 and 180 cm³, between about 190 and 300 cm³, between about 290 to 400 cm³, between about 390 to 500 cm³). For example, a generally spherical scaffold as shown in FIG. 25 may have a belly diameter between about 1 to 7 cm (e.g. about 1, 1.5, 2, 3, 5, or 7 cm). The cage may be made of a biocompatible polymeric knitted mesh made from a material such as polypropylene or polyester. Alternatively, the cage may be made from biodegradable or biodissolvable material such as polylactic-co-glycolic acid (PLGA) or alginate, which may dissolve after a period of time leaving a cavity in the lung parenchyma allowing air to continue venting through the device or allowing the device to be removed. Alternatively, the cage may be made of Nitinol wires, or Nitinol wires coated with a polymer, such as polypropylene or polyester, or a biodegradable polymer. The members of the scaffold (e.g., cage) may be configured to move or deform (e.g., expand, contract) within the lung tissue as the lung tissue moves due to inhalation and exhalation. For example the members of the scaffold may be sufficiently elastic and resilient, or the design of the scaffold structure (e.g., weave, knit, struts, interlocking mechanism, or a wire cage covered with a mesh, weave or knit) may be configured to allow the scaffold to deform in response to forces applied by lung tissue while maintaining a space within the scaffold or maintaining sufficient distance between the lung tissue and the opening(s) 2105 of the air collection tube 2104. In an embodiment having an expandable scaffold comprising Nitinol wire members the wire members may have a diameter in a range of about 0.001″ to 0.015″ (e.g., about 0.003″ to 0.010″) and be weaved or knitted in to an expanded shape such as a spheroid. In an embodiment having an expandable scaffold comprising monofilament polypropylene fibers the fibers may have a diameter in a range of about 0.002″ to 0.015″ (e.g. about 0.004″ to 0.008″). The cage may be configured to have openings or pores 2114, for example between structural members or fibers or wires, to create multiple passageways for air to freely pass from outside of the cage into the cage to allow trapped air in the lung to pass into the cavity created by the cage and then through the tube 2104 and to atmosphere. The pore quantity and size may be configured to reduce tissue ingrowth from significantly obstructing airflow from the lung to the cavity. For example, the pores may comprise a cross-sectional area of about 1 to 100 mm² (e.g., pore diameter may be between about 1-10 mm, or between about 3-7 mm). The cage structure may be configured such that a tissue response results in encapsulation of the cage fibers (e.g., wires) instead of forming bridges to occlude the pore openings. The scaffold may be configured to allow permeation of fluid from lung parenchyma to the air intake component over a period of time consisting of at least 6 months, at least 12 months, at least 18 months, at least 24 months, at least 30 months, at least 36 months, or at least 42 months. A cage could be pre-packaged inside a delivery sheath, and delivered through the access port and be expanded after the sheath is removed. Alternatively, a cage could be self-expanding when a diameter constraining sheath is removed or it may be expanded by inflating a balloon within the cage.

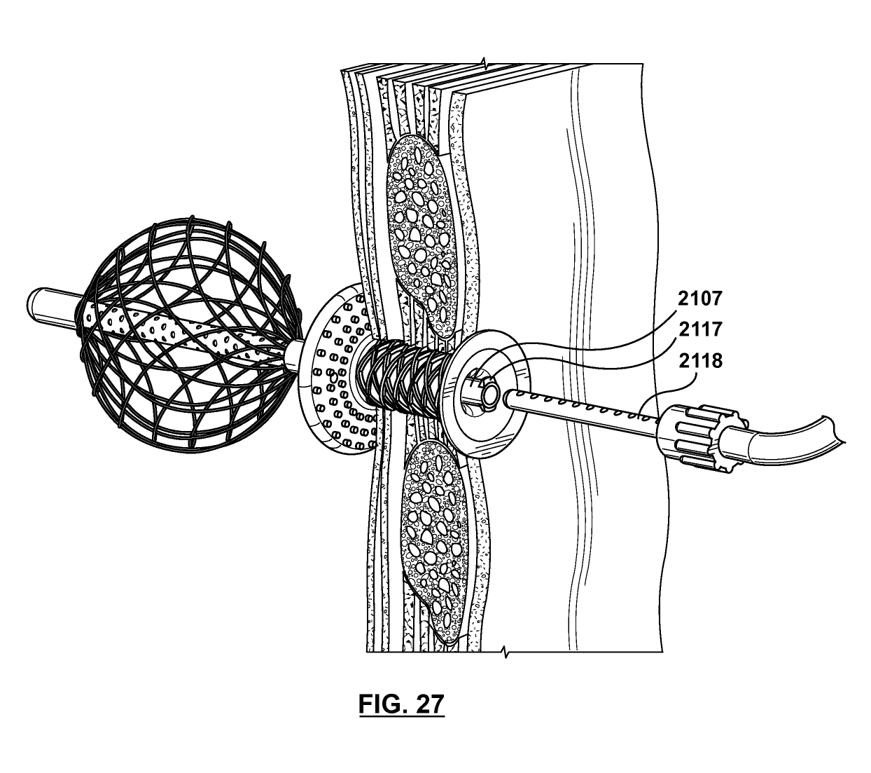

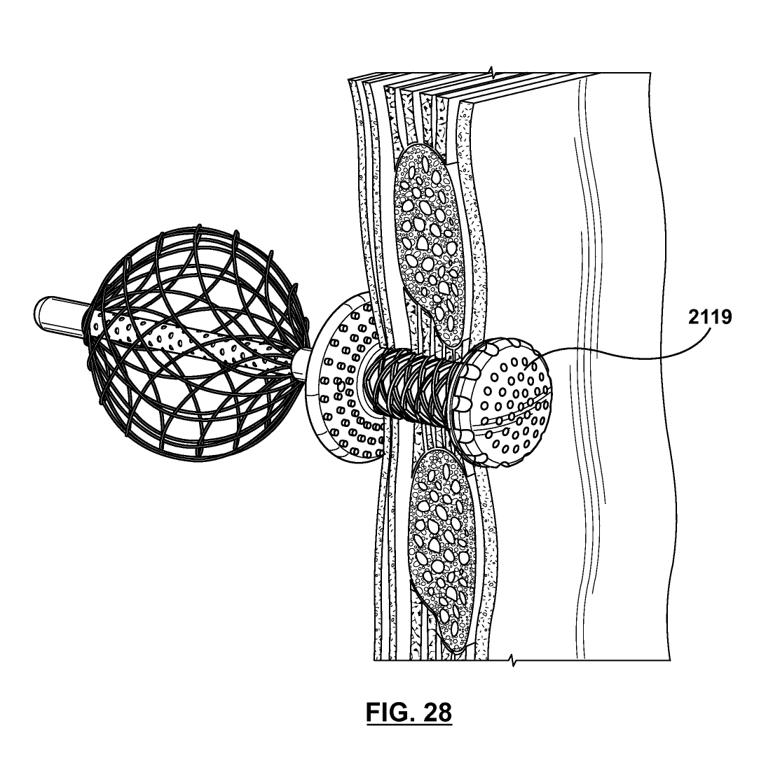

Optionally, an implantable air-venting device may be configured to connect additional components to a portion of the device that is positioned external to the patient. For example, as shown in FIG. 27, the sheath 2107 may comprise a connector fitting on its proximal end, such as a luer adaptor or a clamp adaptor 2117. The connector 2117 may be used to connect components such as a filter, valve, fluid trap container, or plug. FIG. 28 shows a filter unit 2119 connected to the luer adaptor 2117. The filter unit 2119 may further comprise a one-way valve that lets air release from the lung to the environment but not be inhaled through the device. A filter/valve unit 2119 may reduce a risk of environmental contaminants from entering the lung through the device, which may reduce a risk of infection. A filter/valve unit 2119 may be molded from plastic and have a replaceable fabric filter. The connector 2117 may be used to connect instruments used by a physician to clean the vent device such as a drainage system 2118 (FIG. 27) that may be used to aspirate or infuse a fluid, a diagnostic system that assesses airflow properties, a drug delivery system, or a system for delivering gasses such as oxygen. A method of use may involve implanting a port 900, implanting an air collection device 1200, assessing function of the air vent device for example by connecting a diagnostic device to the air vent device, and if proper functioning is confirmed the diagnostic device may be removed and a filter/valve 2119 may be connected. A patient may return to the physician occasionally to clean the device, diagnose function of the device, or administer a drug.

An alternative embodiment of a natural airway bypass system comprises a scaffold that is not expandable but defines a space within the scaffold that is sufficient to maintain a distance between lung tissue and an opening in an air collection tube to prevent tissue from occluding the opening. A port device may comprise a channel having a diameter to allow passage of the scaffold. An air collection tube may be positioned in the space within the scaffold.

Membrane Layers

An expandable structure deployed in lung tissue to maintain space between the lung tissue and an air collection catheter (also referred to as an air intake component) may comprise a membranous layer in addition to a structural layer. When the expandable structure is deployed in lung parenchyma a space or cavity may be created in the lung parenchyma defined by the surface area created by the scaffold and membrane. An air collection catheter resides within the cavity and air passes from lung tissue through membrane orifices to the cavity then through the air collection catheter and out of the body. Alternatively, a cavity in lung parenchyma may be created in a separate step for example by deploying a balloon dilation catheter then deflating and removing the balloon then an expandable structure may be inserted and deployed within the cavity. In either method lung tissue is held away from air passageways of an air collection catheter at least in part by the scaffold or membrane layer.

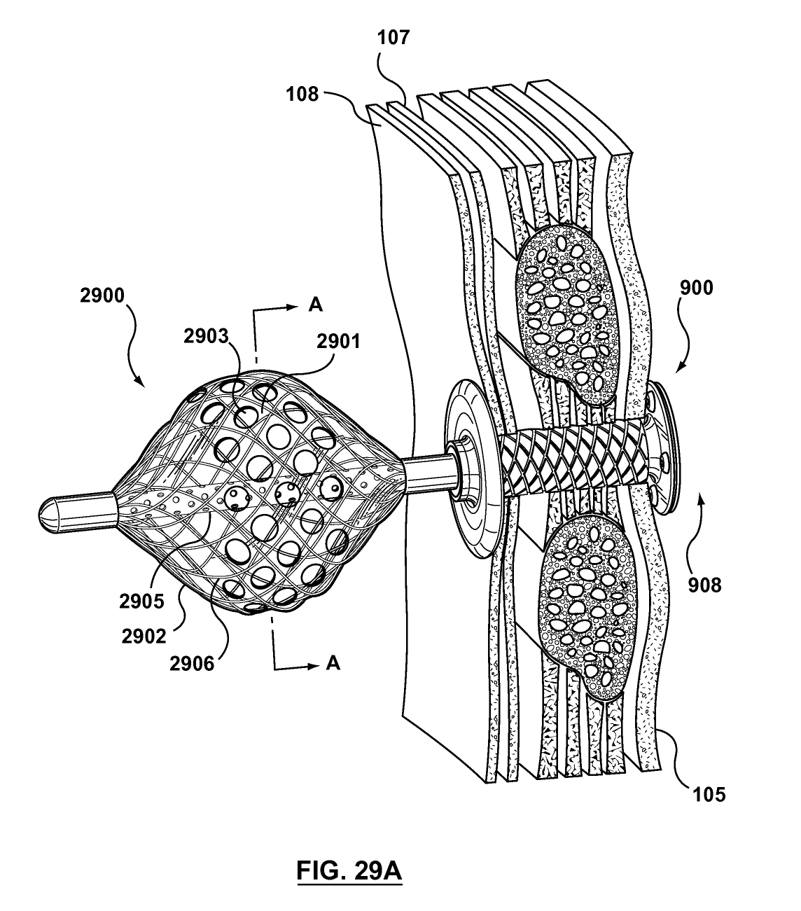

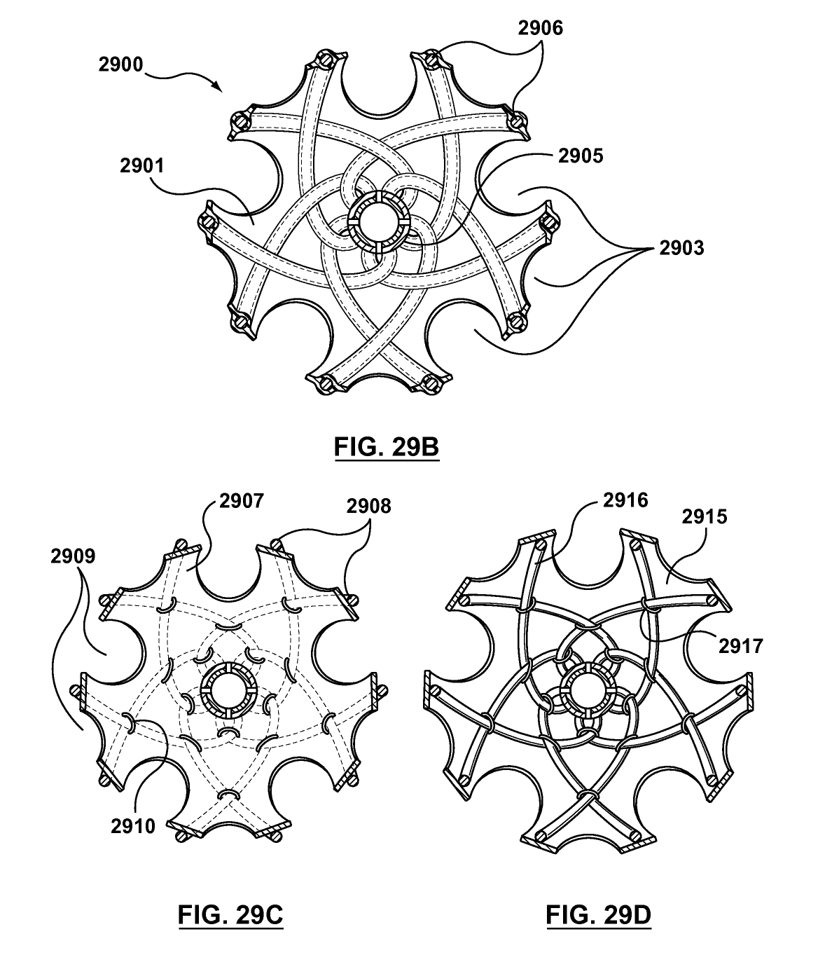

For example, as shown in FIG. 29A, an expandable structure 2900 may comprise a scaffold 2902 of filaments such as Nitinol wires and an integrated membrane layer 2901, the membrane layer having orifices 2903 through which air may pass from lung tissue to a space within the expandable structure before exiting the body through the air collection catheter 2905.

A membrane layer may facilitate deployment of the expandable structure in lung parenchyma. During deployment a membrane layer may reduce tissue injury or irritation by increasing tissue contact surface area as compared to tissue contact with a scaffold without a membrane layer. Increased tissue contact surface area reduces stress concentration or pressure on tissue by spreading force over a larger area. During deployment a membrane may reduce a risk of filaments cutting through tissue instead of pushing tissue away. A membrane may facilitate creation of a cavity within the deployed scaffold and may reduce risk of tissue passing through filaments into the cavity as the cage is deployed.

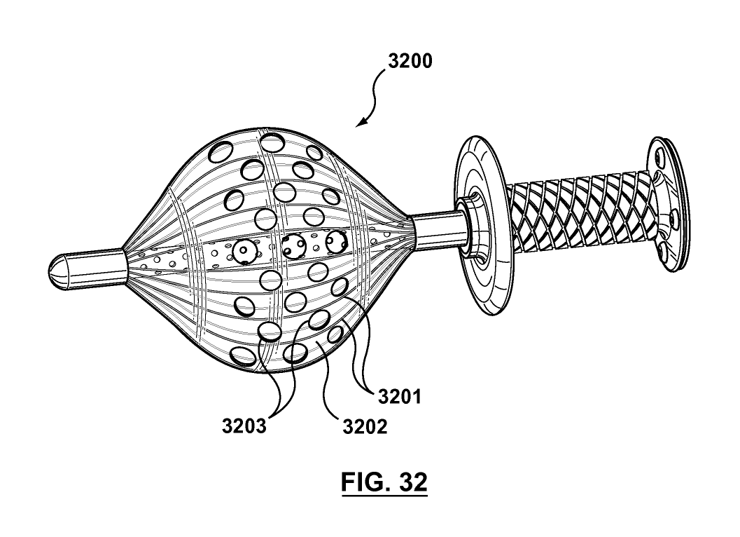

A membrane layer in combination with a scaffold may contribute to the stability of the structural mechanics of an expandable structure. For example a membrane may contribute to structural stability, sheer strength, or hoop strength of a deployed expandable structure, which may further support a scaffold or may relieve some structural function from a scaffold. This is achieved as the covering membrane limits the relative movement of the nearby cage filaments. For example, as shown in FIG. 32 a scaffold 3200 may be configured with relatively few or no intersections of filaments 3201 and a membrane may provide structural strength to maintain a desired shape.

A membrane layer may dampen effects of forceful air movement during coughing or sneezing, particularly if it were an elastic material.

A membrane layer may allow selective control of tissue ingrowth. For example, a membrane layer may inhibit ingrowth of tissue by providing a tissue barrier and promote ingrowth of tissue where there are orifices or where the membrane is not present. A membrane may be used to control ingrowth of tissue in to the space or cavity defined by the expandable structure, or to control attachment of tissue to scaffold filaments. By selectively positioning a membrane layer tissue may be encouraged to attach to an uncovered or uncoated part of a scaffold. For example a membrane layer positioned on an inner surface of a scaffold may promote tissue attachment to the outer surface of the scaffold, which may be desired to control the healing process or secure the device in lung parenchyma or may delay tissue growth or attachment to other parts of the device. In another example, a membrane layer may be configured to inhibit tissue growth in regions of the scaffold that are closest to the air intake component where a risk of tissue growth bridging to the air intake component may be greatest.

A membrane layer may facilitate removal of an expandable structure. For example, a membrane layer may cover the outside surface of a scaffold structure and be made from a material such as silicone that inhibits tissue attachment or increases lubricity so the expandable structure can be contracted to an undeployed configuration and removed from the lung without pulling on tissue that otherwise may have attached to or entangled in the scaffold.

A membrane layer may facilitate cleaning or maintenance of the device. For example, a membrane made from a lubricious material such as silicone, or a non-stick polymer compound such as Endexo® may more easily shed mucus or other debris allowing it to pass through the air intake component and out of the body instead of clogging air pathways in or around the device.

As shown in FIG. 29A, a membrane layer may cover an entire scaffold structure to create a barrier between the scaffold structure and lung tissue. Membrane orifices may be aligned with and smaller than open cells between scaffold filaments. FIG. 29B shows cross-section A-A of FIG. 29A of the expandable structure 2900 wherein scaffold filaments 2906 are incased in the membrane layer 2901 and membrane orifices 2903 are aligned in open cells between the filaments. In this embodiment the membrane layer completely covers the scaffold filaments 2906 and may be applied, for example, by dip coating a deployed scaffold structure in membrane material such as silicone and once the membrane is cured the membrane orifices may be created by laser or chemical etching or mechanically cutting, or other methods for creating controlled holes. Controlled holes in a membrane may be created after the expandable structure is deployed in a patient's lung tissue for example by inserting a tool with an endoscope through a lumen 2113 and into a space within the expandable structure where a user may be able to see via the endoscope through the membrane layer 2901, which may be transparent. The endoscopic tool may be configured to create holes in the membrane layer where desired, for example, to communicate with channels or airways in the lung tissue or avoid creating holes where they are not desired, for example in areas where there is little air passage or where there is substantial blood flow or fluid accumulation. Alternatively, a first membrane layer may be positioned on an inner surface of a deployed scaffold structure, a second membrane layer may be positioned on an outer surface of the deployed scaffold structure and the first and second layers may be bonded together (e.g., thermal bonding, adhesive) around the scaffold filaments. In embodiments wherein a scaffold structure is entirely coated by a membrane layer, tissue will not contact the filaments and may be inhibited from growing around or attaching to the filaments 2906.

FIG. 29C shows an embodiment having a membrane layer 2907 positioned only on an inner surface of a scaffold structure. In this figure the membrane layer is connected to scaffold filaments 2908 with sutures 2910, however other methods of connection may be used. Embodiments wherein a membrane layer is positioned on the inner surface of a scaffold may promote tissue attachment to the filaments 2908 or to the outside surface of the filaments and an air cavity may be maintained around an air collection device by the membrane. Tissue attachment to the scaffold may beneficially allow the scaffold to get integrated with tissue or may allow tissue healing processes to complete so the tissue interfacing with the device is not irritated, or is irritated less than tissue (e.g., granulation tissue) going through a healing process.

FIG. 29D shows an embodiment having a membrane layer 2915 positioned only on an outer surface of a scaffold structure. In this figure the membrane layer is connected to scaffold filaments 2916 with sutures 2917, however other methods of connection may be used. Embodiments wherein a membrane layer is positioned on the outer surface of a scaffold may inhibit tissue contact with the filaments, as tissue will mainly contact the membrane layer. During deployment the filaments may push against the membrane layer which pushes against the tissue. The force applied by the filaments may be spread over a larger area of tissue by the membrane, which may facilitate creation of a cavity in lung tissue or reduce a risk of iatrogenic injury.

The membrane layer 2901 may be thin film made separately and attached to a scaffold structure. Methods of manufacturing the membrane layer may include techniques known in the art such as thermoforming, dip coating or molding to create a specific shape to match the shape of the scaffold in its deployed and undeployed configurations. Alternatively, a membrane layer may be cut from a sheet of film and fabricated (e.g., sewn) in to a desired shape. Alternatively, a membrane layer may be formed directly on at least a part of the scaffold, for example, using techniques such as injection molding or vapor deposition. Materials used to fabricate a membrane layer may include biocompatible materials such as silicone, PTFE, EPTFE, Parylene, a biodegradable material or a combination of materials. A membrane layer may comprise multiple layers or sections. For example, a first layer may be positioned on an inner surface of a scaffold structure and a second layer may be positioned on an outer surface of a scaffold structure. A membrane layer may be thin (e.g., in a range of about 0.002″ to 0.009″) and sufficiently flexible and durable to deform to and from an undeployed state to a deployed or expanded state. Optionally, a membrane layer may be stretchy. Optionally, a membrane layer may be configured to deliver a drug or fluid, which may inhibit infection, control tissue healing, clean the device or treat the lung. For example a membrane may contain a drug in a reservoir and slowly release the drug through pores. A membrane may comprise a lumen through which a drug may be injected from outside the body. A membrane may be impregnated with a drug that is released as the membrane layer biodegrades. Multiple biodegradable drug impregnated membrane layers having different degradation profiles may release a drug at a desired rate based on the degradation profiles.

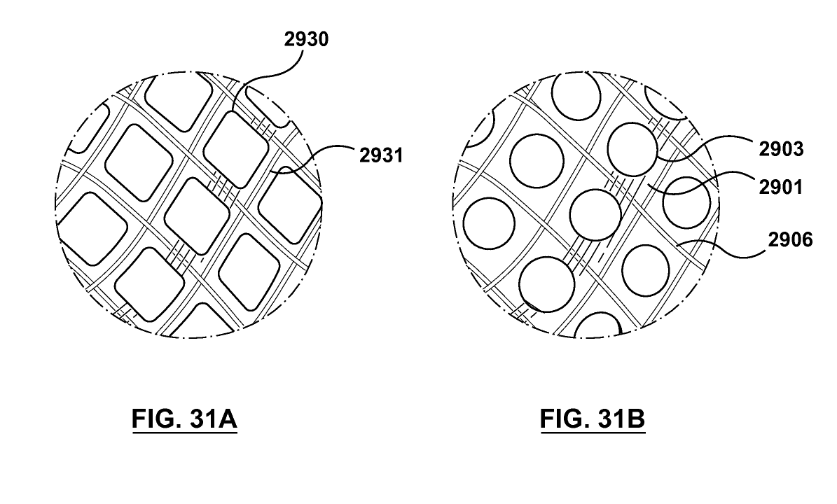

Membrane orifices may have a size and geometry that inhibits tissue ingrowth that may grow over the orifices or inhibits clogging, or provides sufficient flow of air from lung tissue around the device through the orifices to substantially release trapped air. Membrane orifices 2903 may be substantially round as shown in FIG. 29A and FIG. 31B and positioned in openings between scaffold filaments 2906. Round orifices may help to avoid tissue contact with sharp corners, which may reduce irritation and facilitate control of tissue healing. Other shapes of membrane orifices 2930 may be suitable such as a shape similar but offset to the shape of an opening between filaments 2931 and with rounded corners as shown in FIG. 31A. Membrane orifices may have a diameter in a range of about 2 mm to 6 mm, or an area of about 3 mm² to about 29 mm². A membrane layer and scaffold structure may be configured so only one membrane orifice is positioned within a single opening between filaments as shown in FIGS. 31A and 31B. Alternatively, multiple membrane orifices may be positioned in a single opening between filaments as shown in FIG. 32 wherein two or three membrane orifices 3203 are positioned in a space between adjacent filaments 3201. A membrane may have orifices with a variety of shapes and sizes.

A membrane layer may be connected to a scaffold structure for example by suturing multiple filaments or filament intersections to a membrane, by suturing a membrane to proximal or distal ends of a scaffold, by dip coating, by vapor deposition, by inset molding, or with adhesive. Alternatively, a membrane may surround a scaffold structure without being connected to it.

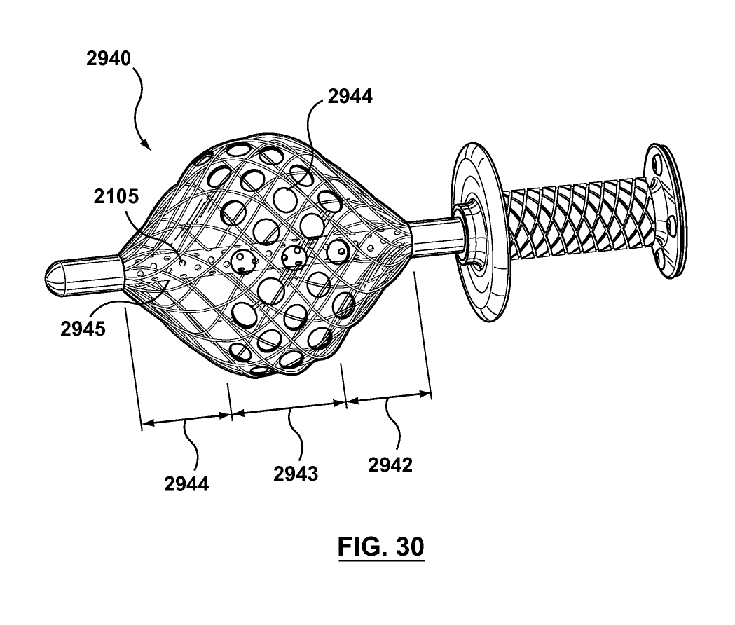

A membrane layer may have orifices positioned in selected regions of an expandable structure. As shown in FIG. 30 an expandable structure 2940 may comprise a distal region 2941, a proximal region 2942 and a middle or belly region 2943. In this embodiment membrane orifices may be positioned only in the belly region that has a surface that is furthest from air passageway(s) 2105 of an air collection device 2945, which may reduce a risk of tissue ingrowth reaching the air collection device, and that may have larger openings between scaffold filaments compared to the distal and proximal regions. The membrane layer around the distal 2941 and proximal 2942 regions may be without orifices to inhibit tissue contact or ingrowth around portions of the scaffold structure where filaments may be closer together or where there may be more filament intersections per unit area.

In another embodiment (not shown) membrane orifices may be positioned predominantly in a caudal direction so fluid in lung tissue is less likely to flow with gravity into the space within the expanded structure when a patient is upright, or fluid that made its way into the space may drain out of the orifices. Alternatively, membrane orifices may be positioned in anatomical directions other than cranially so they are aimed downward if a patient is upright or lying down.

In another embodiment (not shown) membrane orifices may be positioned predominantly toward or in a distal region 2941 so if tissue continues to grow in to the space within the expandable structure and bridges to an air collection device clogging holes in the distal region of the air collection device, holes in a proximal region of the air collection device may remain unclogged for a longer duration. This may increase the duration that the device can effectively allow trapped air to be removed without getting clogged by tissue ingrowth.

In another embodiment (not shown) an expandable structure may comprise two membrane layers, one made from a material that is not biodegradable such as silicone and a second made from a biodegradable material such as PGLA. The first non-biodegradable membrane layer may comprise multiple sets of orifices (e.g., two sets of orifices). A first set of orifices may be open at an early period after implantation to allow trapped air to flow from the lung through the first set of orifices. The second set of membrane orifices may be initially covered by the biodegradable membrane layer. Over time the first set of membrane orifices may get occluded by tissue growth or mucus and the biodegradable membrane layer may dissolve to reveal the second set of membrane orifices so trapped air can continue to pass through the device and out of the lung. This may extend the duration of effectiveness of the device. A similar embodiment may comprise more than two sets of orifices that are revealed sequentially as biodegradable layers dissolve. For example, an embodiment may comprise multiple biodegradable layers having different degradation profiles. The multiple membranes may contain pharmaceutically active drugs, such as anti-inflammation drugs, chemo-therapeutic drugs, and tissue healing growth factors. The multiple layers of membranes may provide controlled drug release at given time periods.

In another embodiment (not shown) an expandable structure may comprise multiple layers of membranes, one membrane to inhibit tissue growth (e.g., silicone) positioned on the inner surface of the expandable structure's shell, and another membrane to encourage tissue attachment (e.g., a porous PTFE) positioned on an external surface of the expandable structure's shell.

In another embodiment an expandable structure may comprise a balloon-like structure having orifices for air to pass from lung tissue to a cavity within the expandable structure. The balloon-like structure may be deployed and maintained in a deployed configuration without a scaffold structure but instead by other methods such as hydrostatic pressure created by injecting saline into lumens in the balloon wall.

Inflatable Anchor Catheter within a Stent

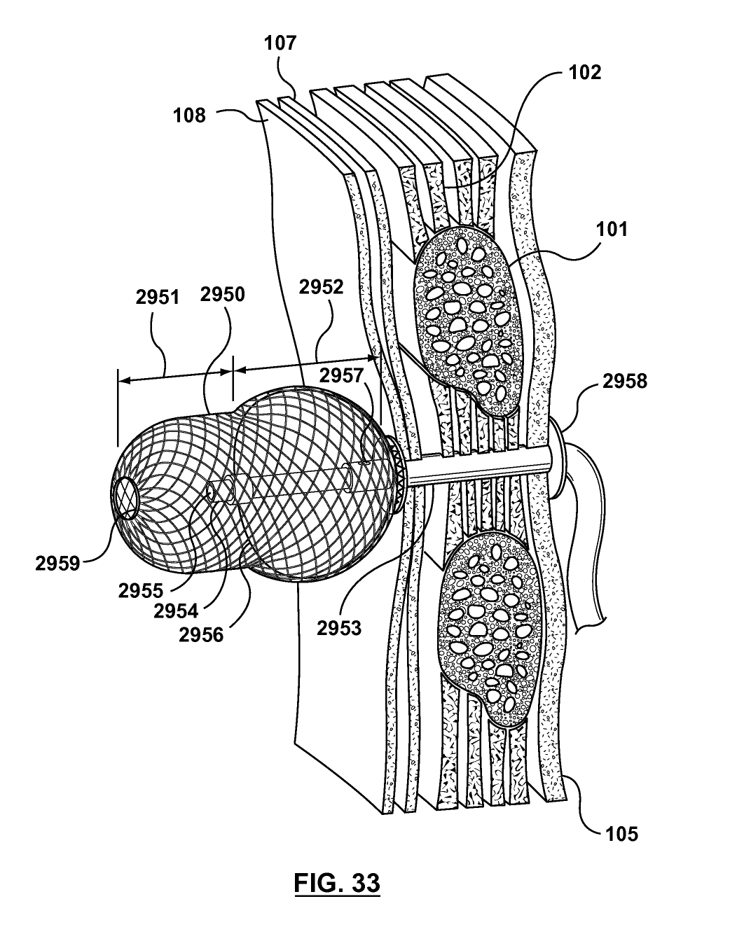

Another embodiment of a system for releasing trapped air in a lung to atmosphere through the chest wall comprises an air collection catheter having an inflatable balloon that is positioned in a stent that is deployed in lung tissue. As shown in FIG. 33 an airway bypass device may comprise an expandable structure comprising a scaffold structure such as a stent 2950 used to maintain a space or cavity in lung tissue, and an air collection catheter 2953 having a tube 2954 with at least one opening 2955 positioned in the cavity. The expandable structure may further comprise a membrane layer (not shown) with orifices. The membrane layer and orifices may be configured as described in other embodiments such as those illustrated by FIG. 29A. The air collection catheter 2953 comprises an elongated tube 2954 configured to pass through a chest wall of a patient between adjacent ribs, a distal section to be positioned in lung tissue, a proximal section to be positioned exterior to the chest wall, an inflatable balloon 2956 positioned on the distal region of the air collection catheter, a lumen 2957 communicating between an interior space of the balloon and the proximal section of the air collection catheter for inflating the balloon (e.g., with saline), and a lumen 2955 communicating between the distal and proximal sections for passage of trapped air from the lung to atmosphere through the air collection catheter. The balloon 2956 may be somewhat spheroid in shape with the tube 2954 passing approximately through its center. The stent 2950 may comprise a balloon-mating section 2952 configured to fit snugly around the balloon 2956, and a cavity-maintaining section 2951 configured to maintain a cavity in the lung tissue around the air collection catheter opening(s) 2955. The device may further be configured to hold the opening 2955 in the cavity away from lung tissue to inhibit tissue growth from occluding the opening. The device may comprise clamp 2958 that may be positioned on the proximal section of the air collection catheter. In use, the tissues of the chest wall may be compressed between the balloon 2956 or balloon-mating section of the stent 2952 and the clamp 2958 to hold the device in place or to apply pressure between the parietal and visceral pleurae to create a pleurodesis. The embodiment shown in FIG. 33 further comprises an opening 2959 on the distal end of the stent 2950, which may facilitate an option of delivering the stent over a guide wire or guide catheter. The at least one opening 2955 in the air collection tube may align with the opening 2959 in the stent allowing both the stent and air collection catheter to be delivered over a guide wire or guide catheter (e.g., the same guide wire or guide catheter). The openings 2955 and 2959 may also facilitate delivery of a catheter such as an endoscope through the device to lung tissue for assessment of the tissue or device.

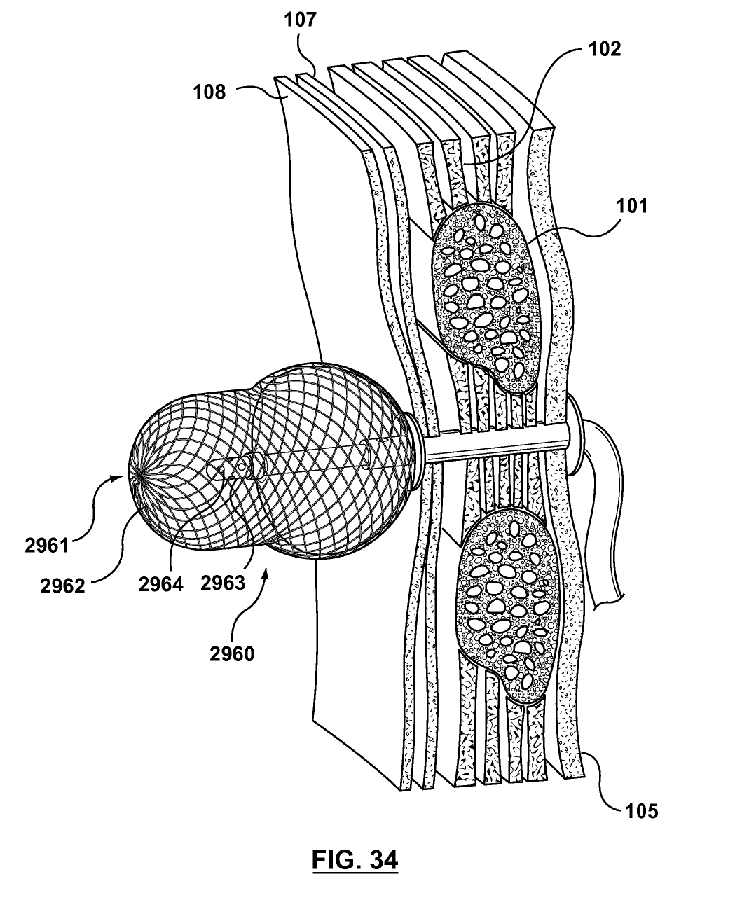

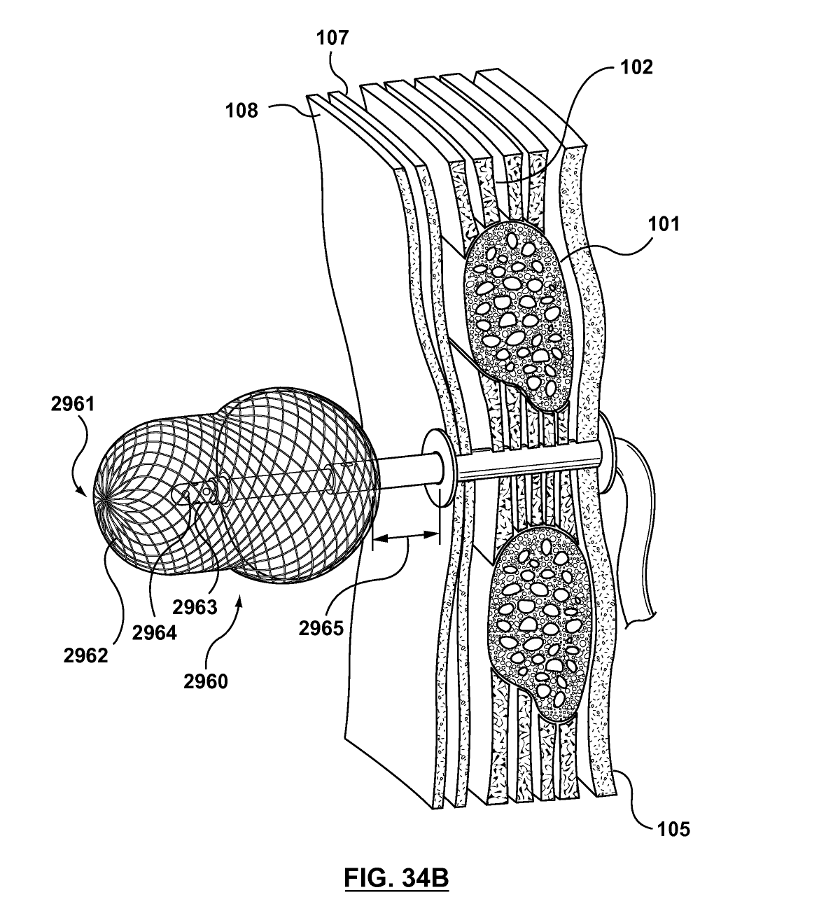

An alternative embodiment shown in FIG. 34A does not comprise an opening 2959 on the distal end of the stent for delivery over a guidewire as shown in FIG. 33. Instead the distal end 2961 of the expandable structure 2960 may be closed off to the tissue, for example with scaffold filaments 2962 or a membrane layer (not shown). The distal region of the air collection catheter 2963 may have at least one opening 2964 into the space inside the expandable structure that doesn't necessarily need to be on the distal tip but may be on the side as shown.

A method of use of the device of FIG. 33 may include the following steps: make an incision in the skin in a location on a patient's chest wall where a natural airway bypass device is to be implanted; optionally cut to the parietal pleura and create a localized pleurodesis; insert a needle, dilator or cannula with a peel-away catheter through the chest wall and in to the lung (e.g., the peel away catheter may have a diameter less than about 12 FR to fit between adjacent ribs in most patients); optionally using a balloon catheter inserted through the peel-away catheter dilate a space or cavity in lung parenchyma having a roughly spheroid shape with a diameter of about 3 cm (e.g. about 1 to 7 cm, about 1, 1.5, 2, 3, 5, or 7 cm) in the area where the natural airway bypass device is to be implanted and remove the dilation catheter; optionally implant a port device 900 such as the one shown in FIG. 29A through the peel-away catheter and remove the peel-away catheter (the design shown in FIG. 33 may optionally be implanted without a port device); insert a stent 2950 delivery system through the peel-away catheter or port device, the delivery system comprising a guide catheter, a collapsed stent 2950 slidably engaged over the guide catheter, and a sheath slidably engaged over the collapsed stent; deploy the stent 2050 by retracting the sheath and remove the stent delivery sheath (optionally tethers or sutures may be tied to the stent 2950 at it's proximal end and the tethers may be positioned through the chest wall and accessible from outside the body), (optionally a tube may be attached to the proximal end of the stent 2950, the tube may be positioned through the opening in the chest wall, and the air collection catheter may be delivered through the tube); insert an air collection catheter 2953 through the chest wall and into the deployed stent (e.g., through the peel-away catheter or over the guide catheter that remains through the stent); deploy the balloon 2956 by injecting saline through lumen 2957 to lock the balloon in the balloon-mating section 2952 of the stent; gently pull the proximal portion of the air collection catheter to apply pressure on the visceral pleura; remove the peel-away introducer catheter; if tethers are tied to the stent they may be tied to the proximal portion of the air collection catheter that remains outside the body; remove the guide catheter or guide wire if it is used; apply a clamp 2958 to the air collection catheter 2953 to maintain pressure and secure the device in place; secure the air collection catheter to the skin; apply skin dressing and treatment around the device.

Alternatively an inflatable balloon anchor may be inflated with a gel or a gel that cures in place to transition from a low viscosity suitable for injecting through a narrow lumen, to a high viscosity or even solid configuration. The cure-in-place substance may be for example a biocompatible epoxy that cures when mixed or a time curing substance or a material that cures in the presence of UV light, which may be applied through a fiber optic to initiate curing. A cure-in-place substance may reduce a risk of an inflatable anchor leaking, which may reduce its effectiveness as an anchor or seal or which may unintentionally deliver the inflation material to the lung. A cure-in-place inflation material may cure slowly enough to allow a user to inflate the anchor, assess if the anchor is positioned and functioning satisfactorily and a user may deflate the anchor by removing some of the inflation material if it is desired to adjust position of the anchor and redeploy it.

As shown in FIG. 34B the expandable structure 2960 may be positioned a distance 2965 away from the chest wall and the air collection catheter 2963 may also function as a flexible tether that allows the expandable structure 2960 to move with lung tissue as the lung tissue moves with respect to the chest wall. The distance 2965 may be in a range of about 0 mm to 15 mm. The flexible section of the air collection catheter may be made from a flexible durometer polymer, polymer compound, or combination of materials yet with sufficient hoop strength to maintain an open lumen to allow air to flow through it.

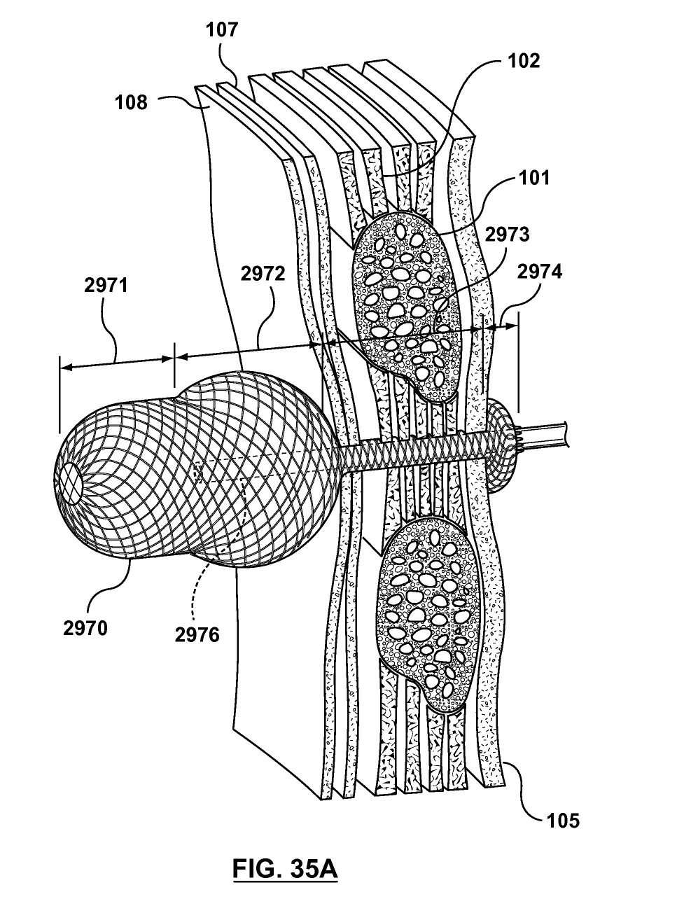

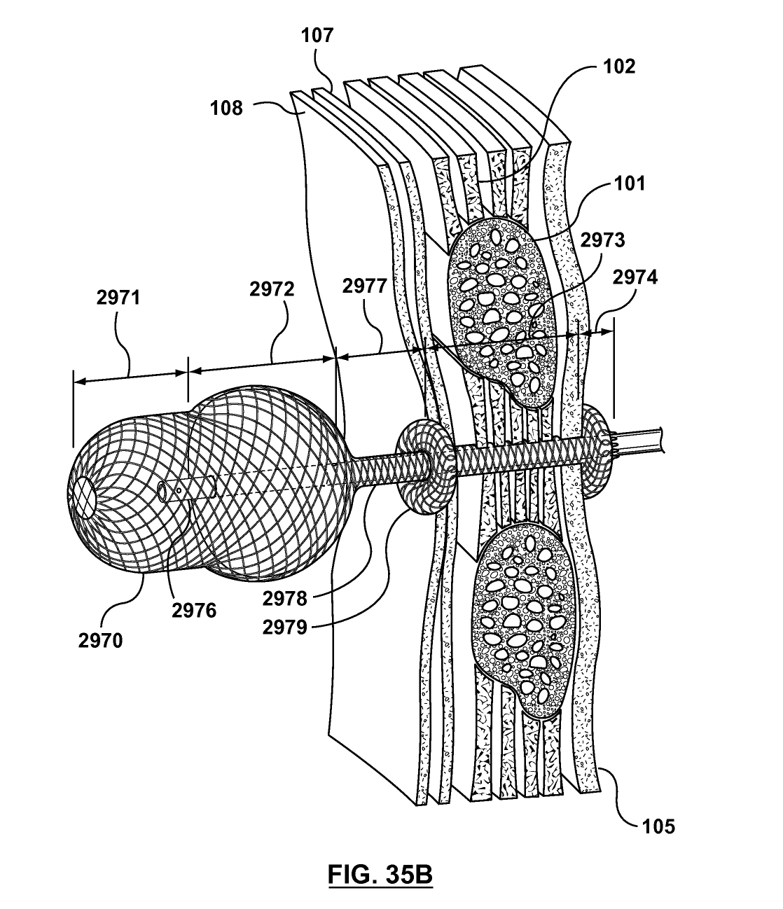

An alternative embodiment shown in FIG. 35A comprises a stent 2970 similar to the stents shown in FIGS. 33, 34A and 34B and further comprising a chest wall section 2973 and an external bulge section 2974. The chest wall section 2973 passes through the tissues of the chest wall. The external bulge section 2974 has a larger diameter than the chest wall section to anchor against the external surface of the chest wall. An inflatable balloon section 2972 may be configured to accept an inflatable balloon that anchors the structure within lung tissue and holds cavity section 2971 open in the lung tissue. The cavity section 2971 of the sent 2970 may be configured to hold lung tissue away from at least one opening in the air collection catheter 2976. In this embodiment the stent may be delivered through a sheath and deployed in a hole through the chest wall without an additional port device. The sheath may be retracted to deploy the stent 2970.

FIG. 35B shows a similar embodiment as the one in FIG. 35A, however the cavity section 2971 and inflatable balloon section 2972 are positioned a distance 2977 away from the chest wall. The distance 2977 may be in a range of about 0 mm to 15 mm. The stent may further comprise an extension section 2978 and an internal anchor section 2979. The internal anchor section 2979 and external bulge section 2974 may hold the stent in place with respect to the chest wall. Furthermore, the chest wall may be gently compressed between the internal anchor section and external bulge section to maintain a pleural seal. The extension section 2978 may position the cavity section 2971 and balloon section 2972 at a distance from the chest wall to allow them to move within the lung tissue with respect to the chest wall, which may improve function or reduce traumatic friction or pressure.

Access Port and Internal Anchor Embodiments

An airway bypass device may comprise a port (e.g. access port or chest wall port) such as the port 900 shown in FIGS. 9 to 25. The port may comprise an internal flange or anchor such as internal flange 905 which may be delivered in an undeployed configuration and then expanded to a deployed configuration on an internal side of the visceral pleura or in the lung. Other embodiments of an internal flange or anchor are described herein.

An internal flange or anchor or an airway bypass device may be configured to apply pressure to a visceral pleura such that the pressure is transferred between the visceral pleura and parietal pleura, which may prevent pneumothorax or create a pleurodesis; create a seal to prevent fluid such as air from passing from the lung (e.g., to a pleural cavity, to space around an implanted port, to tissues external to the lung, to atmosphere); be delivered with minimal or acceptable trauma; be delivered with relative ease and intuitive design. Furthermore, an internal flange or anchor may be configured to function when the tissue contact surface is variable, undulated, or at a variable angle (e.g., within a range of about 45 to 135 degrees) to the port device.

In some embodiments an internal flange be deployed through actuation from a proximal region of the port external to the chest wall, such as the embodiment shown in FIGS. 9 to 25. Alternatively, in some embodiments an internal flange may be self-deploying due to an elastic shape memory design that may resiliently conform to an undeployed state when compressed and advanced through a sheath then expand to a preformed configuration when the sheath is retracted. Deployment of an internal flange or anchor may have benefits such as ease of use, ease of manufacturing and lower cost of manufacturing compared to a system that comprises actuation to deploy such as the embodiment shown in FIGS. 9 to 25 or other embodiments such as an inflatable anchor. A method of implanting a port device in a chest wall may comprise inserting a needle through the chest wall, inserting a guidewire through the needle, removing the needle, inserting a dilator or set of dilators over the guidewire, inserting a sheath, delivering a port device through the sheath and once the port device is positioned in a desired depth in the chest wall the sheath may be removed. As such, the sheath may function as a delivery conduit for the port device and also to maintain a self-deployable internal flange or anchor in an undeployed configuration as it is delivered through the chest wall. When the sheath is retracted the internal flange or anchor may deploy to an expanded configuration, the port device may be repositioned (e.g., pulled outward to apply pressure from the deployed internal flange or anchor to the visceral pleura). Then the sheath may be fully removed allowing tissue of the chest wall to collapse on to the port device, which may comprise a tissue interface such as a Dacron™ sheath. Since a sheath may already be used to deliver a port device its additional function of containing and deploying an internal flange or anchor may reduce additional steps or complexity required compared to embodiments in which other additional components or steps are required to deploy an internal flange or anchor.

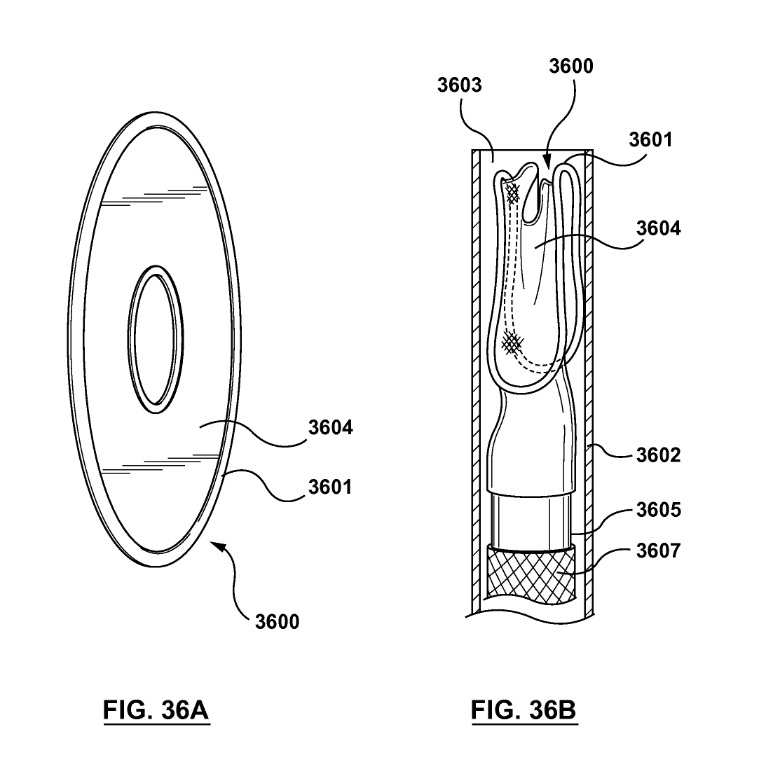

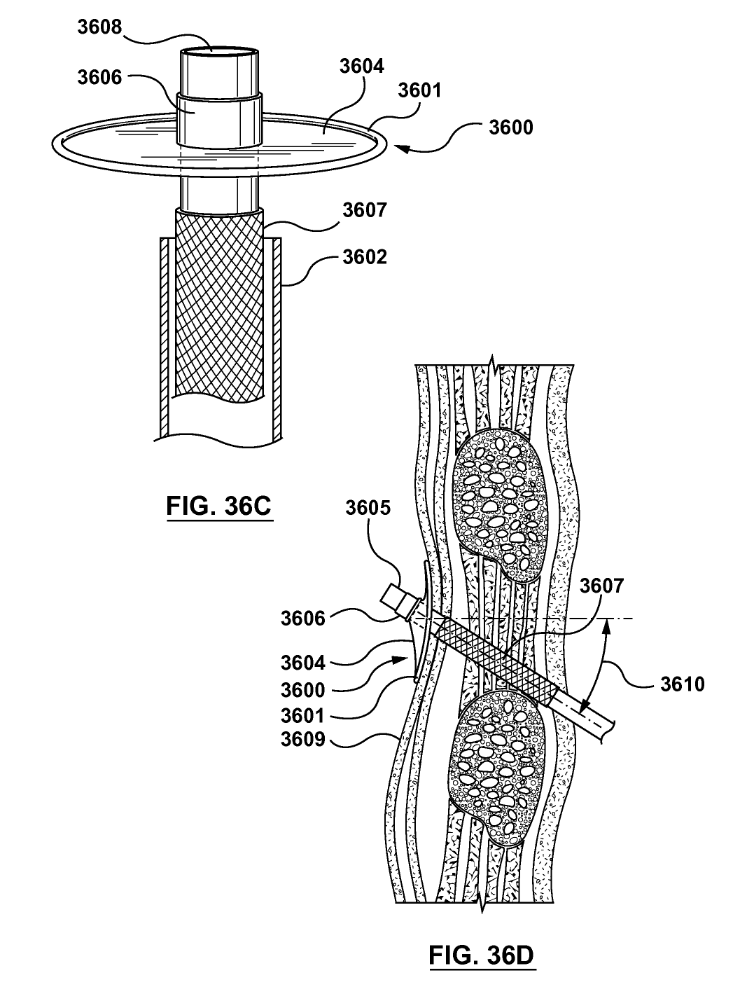

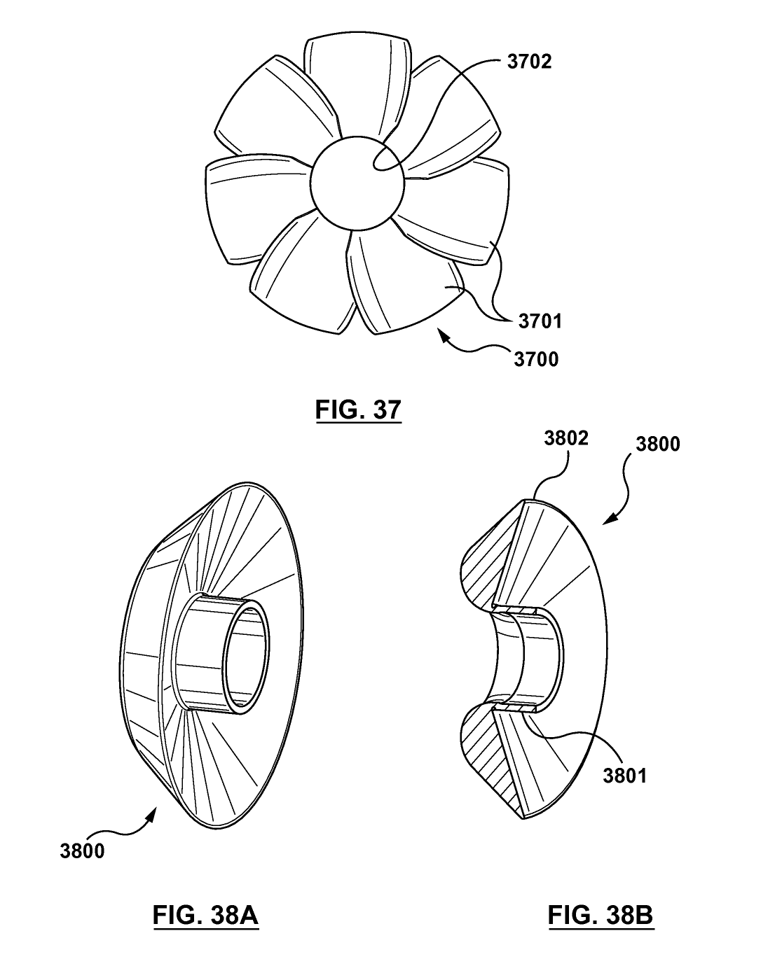

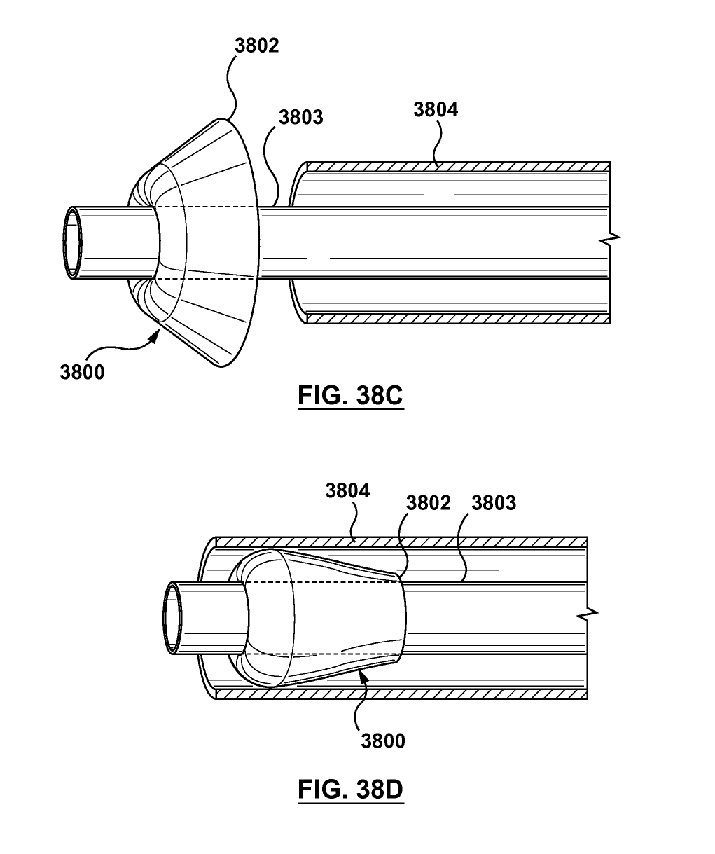

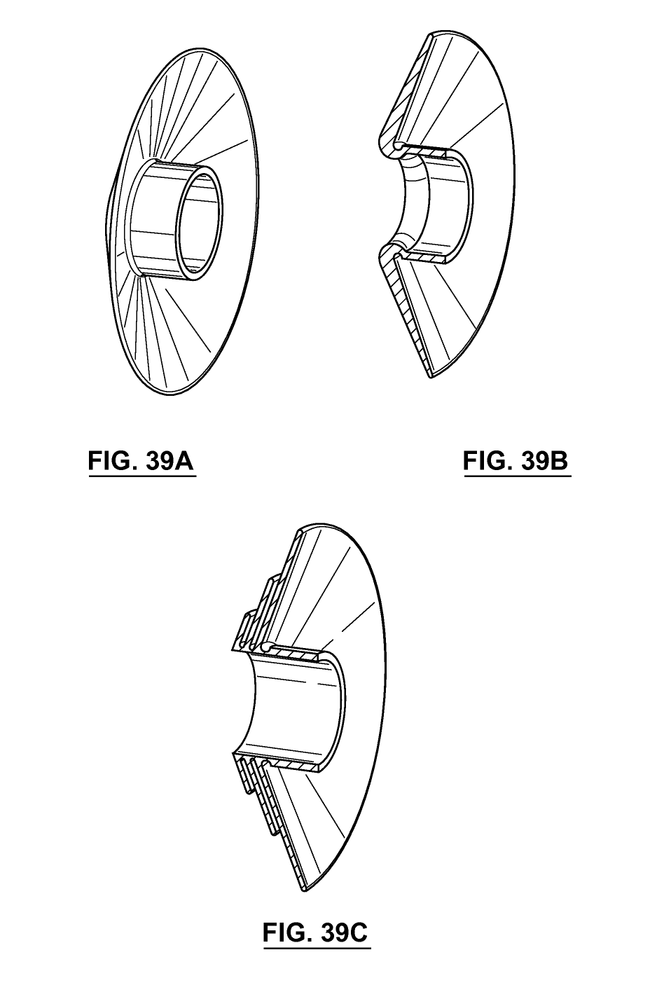

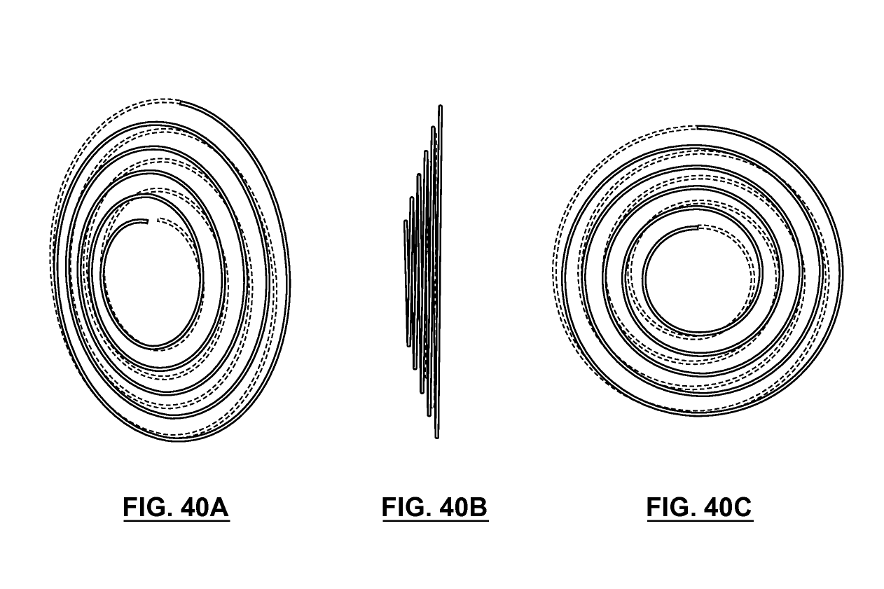

A port and internal flange may be configured to minimize a need for over-travel for deployment. For example, some embodiments of an inner flange such as the inner flange 900 shown in FIGS. 9 to 25 may require the flange to be inserted into the lung at least a distance equal to the flange's undeployed length as shown in FIG. 10. When the internal flange is transitioned to a deployed configuration its diameter is increased whereas its length is decreased as shown in FIG. 11. Over-travel may be defined as the ratio of lengths of an undeployed flange compared to a deployed flange. In some situations it may be desired to minimize the flange's undeployed length or minimize over-travel for deployment. For example, it may be desired to deploy an inner flange or anchor within a COPD void in lung tissue and minimize interruption or trauma to healthy lung tissue that might be caused by over-travel for deployment. An inner flange having a radially expanding conical coil (FIGS. 40A to 40C), expanding foam cone (FIGS. 38A to 38D), elastic cone (FIGS. 39A to 39C) disk (FIGS. 36A to 36D), petals (FIGS. 37A and 37B, FIGS. 41A to 42D) are examples of embodiments that minimize over-travel for deployment.

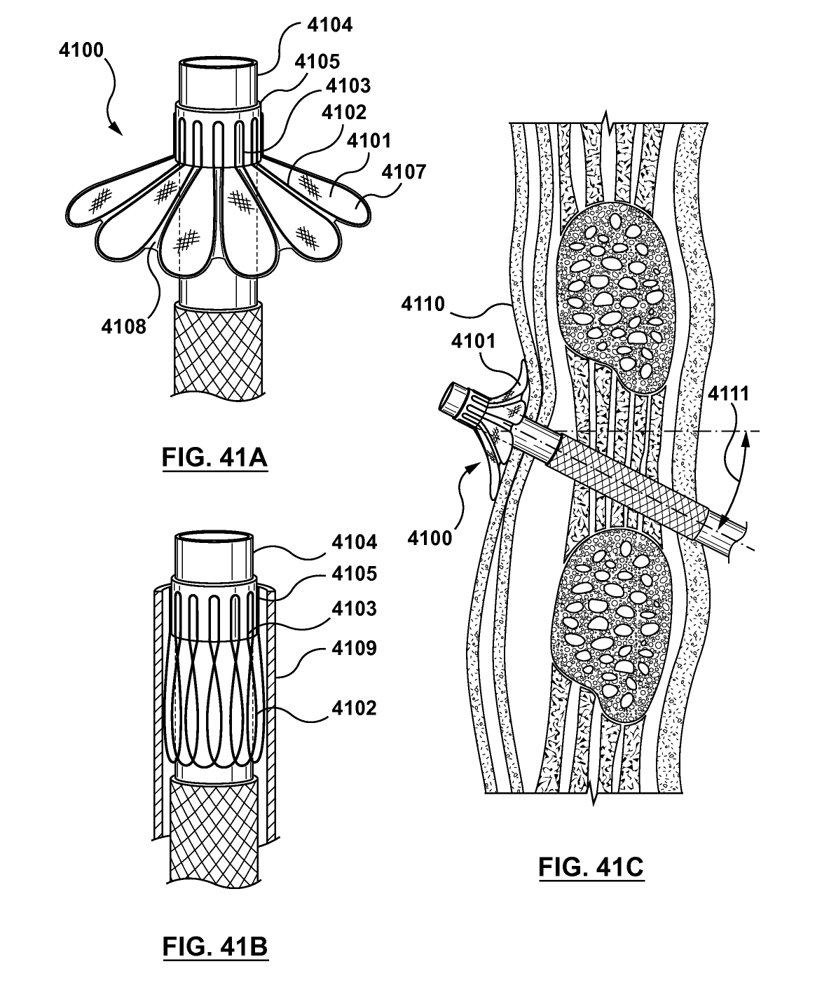

The internal surface of the visceral pleura and anatomical structures such as the ribs may create a surface for contact with the internal flange that is not flat or planar and the surface may be variable. A port and internal flange or anchor may be configured so that the internal flange or anchor conforms to an undulating surface topography to create a seal and apply pressure substantially evenly over the contacting surface or at least around a full circumference of the flange or anchor. An internal flange or anchor may be made of conformable materials such as expanding foam (e.g. FIGS. 38A and 38B), or comprise an elastic material such as a Nitinol® wire or a spring around its circumference (e.g. FIGS. 36A and 36B), or have multiple radial members that independently apply force around a circumference (e.g. FIGS. 37A and 37B and FIGS. 41A and 41B).

The port may be positioned at an angle that is not substantially perpendicular to the internal surface of the visceral pleura where the internal flange or anchor is seated. For example, the angle of the port may vary in a range between about 45 to 135 degrees to the surface of contact of the internal flange or anchor. Thus, a port and internal flange or anchor may be configured to conform to the angle of a port within this range while maintaining the ability to seal and apply pressure to the visceral pleura at least around a full circumference of the internal flange or anchor. For example, embodiments shown in FIGS. 36A to 42D comprise an internal flange or anchor that may be self-deployed by retraction of a delivery sheath and effectively apply pressure to an inner surface of a chest wall when a port device is delivered over a varying range of angles (e.g. between 45 to 135 degrees to the surface).

Some embodiments may comprise an internal flange or anchor that may be retracted or redeployed. For example, if an attempt to deploy the internal flange and create a seal is not satisfactory the internal flange or anchor may be partially or fully transitioned from an expanded deployed configuration to a contracted undeployed configuration and deployment may be reattempted. This may be accomplished for example by pushing the delivery sheath back over the internal flange or anchor which may bend or compress it to reduce its radius. Reseating an internal flange or anchor may alternatively or additionally comprise manipulation of the port device or internal flange or anchor by rotation or adjustment of depth into a lung or chest wall.

An embodiment of an internal flange or anchor may comprise a self-deployable disc-shaped flange as shown in FIGS. 36A, 36B and 36C. The disc 3600 may comprise a spring 3601 such as a coiled spring or a super-elastic Nitinol wire having a preformed shape such as a circle with a diameter that is larger than the diameter of the opening in the chest wall or the sheath. For example a lumen 3603 in a sheath 3602 may have a diameter in a range of about 2 to 5 mm and a diameter of a circle formed by the spring 3601 may be in a range of about 5 to 20 mm. A flexible membranous material 3604 such as EPTFE or silicone or a polymer compound such as Endexo® may form a surface of the disc 3600 and be connected to the spring 3601 and the port tube 3605. As shown the membrane is connected to the port tube with a collar 3606. FIG. 36B shows a disc-shaped internal flange 3600 in an undeployed configuration within a delivery sheath. The spring and membrane may be folded to fit in the lumen 3603 of the sheath and may be positioned distal to the port tube 3605 within the delivery sheath as shown. Alternatively a disc-shaped internal flange may be folded around a port tube when placed in a delivery sheath. FIG. 36C shows the disc-shaped internal flange in a deployed configuration with the delivery sheath 3602 retracted. The elastic force of the spring 3601 encourages the disc to unfold toward its preformed shape. Further retraction of the delivery sheath will allow the tissue of the chest wall to collapse around the port tube 3605. Optionally, a tissue interface texture or component 3607 may encourage tissue of the chest wall to adhere or grow into the tissue interface, which may provide controlled tissue healing. FIG. 36D shows a port device comprising a disc-shaped internal flange 3600 implanted in a chest wall wherein the internal flange 3600 conforms to a non-planar, undulating, curved surface 3609 and wherein the internal flange 3600 conforms to the surface 3609 when the port tube 3605 is positioned in a chest wall at an angle 3610 that is not perpendicular to the surface 3609.

FIG. 37 shows an embodiment of an internal flange or anchor 3700 having multiple petals 3701 in a deployed configuration. The multiple petals may be independently connected to the port tube at the petals' neck 3702 which may have elastic properties that encourage the petals to deploy to an opened configuration when a delivery sheath is retracted and allow the petals to be folded in to a reduced radius undeployed configuration. The multiple petals may allow independent pressure application of each petal on to tissue facilitating use on a non-planar, undulating, or curved surface or positioning at a variable angle.

Another embodiment of an internal flange having independent petals 4100 is shown in FIGS. 41A-C. The petals 4101 are formed with frame 4102 made from an elastic material such as super-elastic Nitinol® that bends in multiple loops having a petal neck segment 4103 that is connected to the port tube 4104 for example with a collar 4105, and a petal segment 4101 that extends from the port tube. A flexible membrane material 4107 such as EPTFE, silicone or a polymer compound such as Endexo® covers the elastic frame 4102 to fill in the petals and optionally to fill in the space 4108 between each adjacent petal, which may further strengthen the internal flange structure or provide a sealing function. FIG. 41B shows the internal flange with petals in an undeployed configuration in a delivery sheath 4109 wherein the petals may bend at the petal neck 4103 and fold down over the port tube and overlap with adjacent petals. In an undeployed configuration the flexible membrane may fold (not shown). FIG. 41C shows the internal flange 4100 with independent petals 4101 pressing on the pleural surface 4110. Independent pressure applied by each petal may facilitate the ability of the flange to conform to an undulating, non-planar, curved surface or when the port tube is positioned at an angle 4111 that is not perpendicular to the surface.

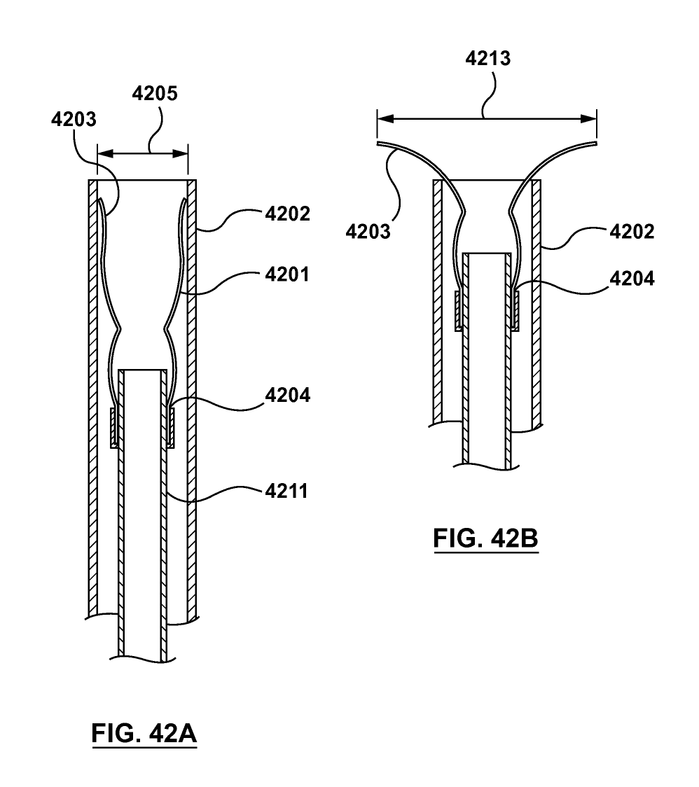

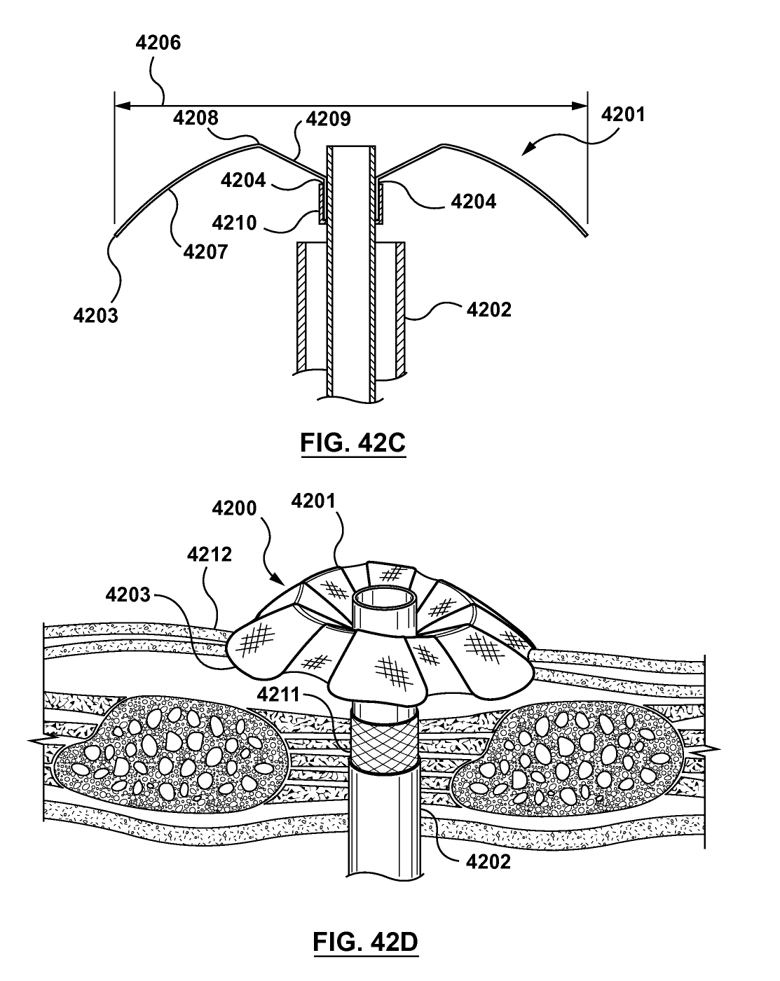

Another embodiment of an internal flange having independent petals 4200 is shown in FIGS. 42A to 42D. In this embodiment the petals 4201 fold distally (distal to the connection with the port tube) when contained in a delivery sheath 4202 as shown in FIG. 42A. In contrast to the embodiment shown in FIG. 41A to 41C distally folding petals may deploy differently and facilitate retraction and reseating. As a delivery sheath 4202 is retracted (FIG. 42B), the distal ends 4203 of the petals may begin to bend out before the sheath 4202 exposes the petal necks 4204 thus gradually expanding the diameter of the internal flange from an undeployed diameter 4205 (FIG. 42A) to intermediary diameters 4213 (FIG. 42B) to a deployed diameter 4206 (FIG. 42C). When the sheath is retracted to a position exposing the necks 4204 of the petals the internal flange 4200 may be fully deployed resulting in no or minimal over-travel. Gradual expansion may be less traumatic to lung tissue compared to exposing the tissue to expansion forces over a brief period. The shape of the petals may further contribute to beneficial deployment features. For example, as shown in FIG. 42C a cross-section of an internal flange having distally folding petals in a deployed configuration, the profile of the petals may comprise a wire frame having a distal curved section 4207, a bend 4208, a substantially straight section 4209, a neck bend 4204, and a connection section 4210. As the sheath 4202 gradually retracts the curved section 4207 may gradually flare outward increasing the flanges diameter gradually; when the bend 4208, straight section 4209, and neck bend 4204 are is released from the sheath the opened internal flange may over extend (e.g., the distal ends of the petals 4203 may be moved in a direction toward the proximal end of the port or toward the pleura). This embodiment may facilitate removal or reseating. A delivery sheath 4202 may be advanced over the internal flange engaging first with the straight section 4209 of the petals to bend them forward then the port device and flange may be pulled into the sheath and collapsed back to an undeployed configuration. FIG. 42D shows a port device 4211 positioned in a chest wall with the internal flange 4200 applying pressure to a visceral pleura which may have a surface 4212 that is non-planar, undulating, or curved or at an angle that is not perpendicular to the surface 4212.

FIG. 38A shows an embodiment of a self-expanding internal flange or anchor component 3800 made from a foam material that may be compressed to an undeployed configuration for delivery through a sheath and may expand toward a preformed shape when compressive forces of the delivery sheath are removed. The expansion of the foam flange may be dependent on forces applied to it for example from lung parenchyma or an inner chest wall surface. Thus the foam internal flange may be compliant or conform to a non-planar, undulating, or curved surface or at varying angles. The foam may expand to fill a small void in lung tissue, or expand applying gentle pressure to lung parenchyma which may suit the functions of minimal trauma and conformation to a surface to effectively create a seal. As shown in FIG. 38B, a cross section of the foam internal flange, the component 3800 may comprise a collar 3801 for connection to a port tube, and a flange having a conical shape wherein the base 3802 of the conical shape is toward the proximal end of the port device where it is intended to apply pressure to the internal surface of the chest wall. The cross section shows that the foam thickness tapers down towards the base 3802. FIG. 38C shows the foam internal flange in a deployed configuration mounted to a port tube 3803 with a delivery sheath 3804 retracted. FIG. 38D shows the foam internal flange 3800 in an undeployed configuration within a delivery sheath 3804. Implantation of a foam internal flange relies on the ability of the foam to recover its uncompressed shape. Long-term compression may impede the ability of the foam to fully expand. Instead of long-term compression, for example providing and storing the flange in a compressed state, the flange may be provided and stored in a deployed state and compressed to an undeployed state when a user is ready to insert it through a delivery sheath during an implant procedure.

Embodiments of a self-expanding internal flange or anchor comprising a conical shape are shown in FIGS. 39A to 39C. These embodiments comprise an internal flange made of a flexible, elastic material such as silicone formed in a conical shape with a base of the conical shape positioned towards the proximal end of the port device or intended to apply pressure to the internal surface of the chest wall. The elasticity of the component may have varying elastic resilience imparted to the device by altering thickness of the material as shown in FIG. 39B wherein the material tapers toward the conical base; or by varying a number of layers as shown in FIG. 39C wherein the flange comprises more layers (e.g., 3) of elastic material towards the center of the component, decreasing to less layers (e.g., 2 then 1) toward the base of the cone.

An embodiment of a self-expanding internal flange comprising a spring mesh in a conical shape is shown in FIGS. 40A to 40C. A spring mesh may be fabricated with a spring wire wound into a coil and a second spring wire wound into a coil in an opposite direction. The two wires may be braided together for example intersections may alternate between over and under lapping. Other configurations of a spring mesh may be envisioned wherein spring wire such as Nitinol® or spring stainless steel is formed into a conical or disc shape or other shape that extends radially from an internal radius where the flange is connected to a port tube. A flexible membrane material may cover the spring mesh.

In addition to an internal flange or anchor, a tissue glue (e.g. lung sealant, a soft tissue glue) may be injected between the internal flange or anchor and the internal surface of the chest wall to enable adhesion at a relatively low contact pressure. A tissue glue may help to maintain a seal even if the compressive pressure applied by the internal flange on the tissue is relieved.

A port device and internal flange or anchor may be configured to allow imaging technology to assist in assessing if the device is implanted satisfactorily. Imaging may also be used during a procedure of implanting the device to facilitate in the procedure. Imaging technology such as x-ray or fluoroscopy may be used to image radiopaque markers placed on the device, for example on a distal region of the port tube or on portions of the internal flange or anchor. In embodiments comprising a collar holding an internal flange to the port tube, the collar may be a radiopaque band. Embodiments having a spring wire incorporated in the internal flange (e.g., wire or spring 3601 of FIGS. 36A to 36D, wire mesh of FIG. 40A to 40C, wire loops 4102 of FIGS. 41A to 41C, or wire loops 4201 of FIG. 42A to 42D) the wire may be radiopaque or radiopaque markers may be fastened to the spring wire for example around the outer circumference of the internal flange (not shown). A radiopaque contrast may be injected to see how it flows in tissue or in or around the implanted device, for example to show if an implanted port device is creating a satisfactory seal.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s).

In this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1.-145. (canceled)

146. An airway bypass device comprising:
a distal portion assembly including a permeable chamber and an air intake device, wherein the permeable chamber is configured to form an air collection space within a lung parenchyma, wherein the air intake device includes an air intake within the air collection space
an outer wall of the permeable chamber configured to displace the lung parenchyma and enclose the air collection space;
an air passage open to the air intake and including an air outlet at a proximal end of the air passage, wherein the air passage is configured to extend from the lung parenchyma and through a chest wall; and
a proximal portion assembly configured to abut or attached to skin over the chest wall, wherein the proximal portion supports a proximal region of the air passage.

147. The airway bypass device of claim 146 wherein the air passage is a tube extending between the air intake device and the proximal portion assembly.

148. The airway bypass device of claim 146 wherein the outer wall is an expandable structure having a greater width than a width of the air passage.

149. The airway bypass device of claim 146 wherein the outer wall structure is a wire mesh.

150. The airway bypass device of claim 146 further comprising a membrane covering at least a portion of the outer wall surrounding a junction between the outer wall and the air intake device.

151. The airway bypass device of claim 146 further comprising a one-way valve coupled to the air passage and configured to allow air to exhaust through the vent.

152. The airway bypass device of claim 146 wherein the air intake device includes an anchor configured to seat on an internal surface of a chest wall.

153. The airway bypass device of claim 152 wherein the anchor is a disc structure extending around a shaft of the air intake device and having a center opening attached to the air intake device and an outer annular flange configured to seat on the internal surface of the chest wall.

154. A device for airway bypass of a diseased lung comprising:
a distal region, and
a proximal region,
wherein the distal region comprises an air intake component and an expandable structure,
wherein the device is configured to hold the air intake component within a space in lung tissue maintained by the expandable structure,
a conduit connecting the air intake component to the proximal region,
wherein a lumen in the conduit fluidly communicates between the air intake component and the proximal region.

155. A device for airway bypass of a diseased lung comprising:
a distal region, and
a proximal region,
wherein the distal region comprises an air intake component and a scaffolding structure, a space defined within the scaffolding structure configured to maintain a distance between lung tissue and an opening in the air intake component,
wherein the device is configured to hold the air intake component within the space,
a conduit connecting the air intake component to the proximal region,
wherein a lumen in the conduit (fluidly communicates between the air intake component and the proximal region.

156. The device according to claim 154, wherein the expandable structure surrounds the air intake component.

157. The device according claim 154, wherein the expandable structure is configured to create the space within the lung tissue and separate the lung tissue from the air intake component.

158. The device according claim 154, wherein the air intake component comprises a port to the lumen in the conduit.

159. The device according to claim 154 wherein the expandable structure comprises a scaffolding structure of struts or fibers or wires.

160. The device according to claim 159, wherein the scaffolding structure is expandable from a thin, undeployed state, and to an expanded, deployed state, having an increased volume.

161. The device according to claim 159, wherein the scaffolding structure is one of a cage, a mesh, a basket, a weave or a stent.

162. The device according to claim 154, further comprising a one-way valve positioned at the proximal region and coupled to the conduit.

163. The device according to claim 154, further comprising a plug positioned at the proximal region of the device, wherein the plug is configured to form a seal around the conduit and hold the conduit with respect to the skin.

164. The device according to claim 154, wherein the expandable structure in fully deployed state is a spheroid with diameter in a range of 1 to 7 cm.

165. The device according to claim 154, wherein the expandable structure has pores each having a cross-sectional area between about 1 to 100 mm².

166. The device according to claim 154, wherein the expandable structure comprises a deployable scaffolding structure connected to a distal end of the air intake, wherein the expandable structure is connected to a proximal end of a sheath such that the air intake component slides telescopically within a lumen of the sheath.

167. The device according to claim 154 wherein the scaffolding structure comprises a membrane layer and an orifice in the membrane layer configured to pass air to pass from the lung tissue to the cavity.

168. The device of claim 167, wherein the membrane layer on an outer surface of the scaffolding structure.

169. The device of claim 167, wherein the membrane layer is formed of a non-stick polymer which inhibits tissue attachment.

170. The device of claim 167, wherein the membrane layer covers the entire outer surface of the scaffolding structure.

171. A method for venting trapped air of a diseased lung comprising:
inserting an artificial passageway through a chest wall and into a lung of a patient;
displacing lung parenchyma in the lung by expanding an outer wall structure of the artificial passageway, wherein the outer wall structure is permeable to the passage of air from the lung parenchyma;
positioning an air intake device within the outer wall structure wherein an air intake port of the air intake device is positioned within a volume formed in the lung parenchyma by the expanding outer wall structure;
positioning an air exhaust vent on skin of the chest wall of the patient and
establishing an air exhaust passage configured to exhaust air from the lung, through the air intake device and out of the exhaust vent.

172. The method of claim 171 wherein the outer wall structure includes a wire mesh and the displacement of the lung parenchyma includes expanding the wire mesh from a compressed configuration in which the wire mesh is adjacent a shaft including the air intake device to an expanded configuration which displaces the wire mesh radially outward from the shaft.

173. The method of claim 171 further comprising implanting within the chest wall an expandable internal flange, expanding and seating the internal flange against the chest wall, wherein the internal flange supports the air intake device within the chest wall.

174. The method of claim 171 further comprising seating an external flange against the skin and connecting the external flange to a tube assembly including a conduit include in the air exhaust passage.

175. The method of claim 171 wherein the tube assembly includes a first tube connected to the internal flange and a second tube connected to the external flange, and the first and second tubes are coaxial and coupled together.

176. The method of claim 171 wherein the first tube and second tube slide one in the other and may be adjusted to form a length of the tube assembly of between 2 cm and 8 cm.

177. The method of claim 171 further comprising applying a polymeric membrane covering the outer wall structure.

178. The method of claim 171 further comprising identifying a lung region of lung parenchyma having one or more characteristics including: low density lung tissue, collateral ventilation channels and a lack of major blood vessels, and the displacement of the lung parenchyma is in the identified lung region.

179. An airway bypass device comprising:
a permeable chamber configured to form an air collection space within a lung parenchyma,
an outer wall of the permeable chamber configured to displace the lung parenchyma and enclose the air collection space;
an air intake within the air collection space;
an air passage having an inlet at the air intake and configured to extend from the lung parenchyma and through a chest wall; and
a proximal housing configured to abut or attached to skin over the chest wall, wherein the proximal housing supports an outlet end of the air passage,
wherein the air passage is configured to vent air from within the air collection space through the chest wall and the proximal housing and into the atmosphere.
